(19) United States
(12) Patent Application Publication
(10) Pub. No.: US 2026/0224242 A1
(43) Pub. Date: Aug. 6, 2026

(54) PUNCTURE NEEDLE

(71) Applicant: TERUMO KABUSHIKI KAISHA, Tokyo (JP)

(72) Inventors: Kenichi HAZAMA, Nagoya city (JP); Yasuhiro KAKIMOTO, Hadano city (JP)

(73) Assignee: TERUMO KABUSHIKI KAISHA, Tokyo (JP)

(21) Appl. No.: 19/629,368

(22) Filed: Mar. 26, 2026

Related U.S. Application Data
(63) Continuation of application No. PCT/JP2024/032505, filed on Sep. 11, 2024.

(30) Foreign Application Priority Data
Sep. 29, 2023 (JP) 2023-170000

(51) Int. Cl. A61B 17/34; A61B 17/00

(52) U.S. Cl. CPC A61B 17/3417; A61B 17/00234; A61B 2017/00296; A61B 2017/3454

(57) ABSTRACT

A puncture needle that enables a liquid medicine to be efficiently injected even in a case where the puncture needle has a small diameter and has high insertability into biological tissue is provided. The puncture needle includes a needle tube portion having a needle tube lumen extending in an axial direction, a cutting edge portion having a plurality of cutting surfaces formed at different positions around an axis and a plurality of ridge lines positioned at respective boundaries of the plurality of cutting surfaces, the cutting edge portion being arranged on a distal side of the needle tube portion; and one or more hole portions that are formed in a side surface of the needle tube portion. The one or more hole portions are arranged on an extension of each of the plurality of ridge lines in the axial direction.

### CROSS-REFERENCES TO RELATED APPLICATIONS

This application is a continuation of International Application No. PCT/JP2024/032505 filed on September 11, 2024, which claims priority to Japanese Application No. 2023-170000 filed on September 29, 2023, the entire content of both of which is incorporated herein by reference.

### TECHNOLOGICAL FIELD

The present disclosure relates to a puncture needle.

### BACKGROUND DISCUSSION

Conventionally, in the medical field, there have been advances in development of puncture needles for realizing local administration of a liquid medicine to a target site in a living body together with puncturing biological tissue.

It is desirable that the puncture needles have a structure having a smaller diameter from the viewpoint of enhancing deliverability to each part in a living body. However, when the puncture needle has a small diameter, a lumen diameter of the puncture needle is reduced, and the injection resistance when a liquid medicine is administered to a target site increases. Therefore, when the puncture needle has a small diameter, a problem arises in that it is not possible to efficiently administer the liquid medicine to the target site. In addition, when the puncture needle has a small diameter, the pushability of the puncture needle is lowered, and thus the insertability into biological tissue is also lowered.

For example, Japanese Patent Application Publication No. 2010-512863 A discloses a puncture needle (puncture instrument) including a cutting edge portion having a plurality of tapered surfaces tapered toward a distal end and ridge lines extending between the tapered surfaces, a side hole arranged at a position displaced from the distal end of the cutting edge portion toward a proximal side, and a liquid medicine administrating lumen communicating with the side hole.

According to the puncture needle of Japanese Patent Application Publication No. 2010-512863 A, since the cutting edge portion is configured to have the plurality of tapered surfaces and the ridge line extending between the tapered surfaces as described above, the biological tissue can be cut and opened by the plurality of tapered surfaces and the plurality of ridge lines when the biological tissue is punctured with the cutting edge portion. Therefore, it is considered that the insertability of the cutting edge portion into the biological tissue is relatively good. However, even when the structure of the cutting edge portion disclosed in Japanese Patent Application Publication No. 2010-512863 A is employed, it can be difficult to avoid an increase in injection resistance when a liquid medicine is administered in a case where the puncture needle has a small diameter. Therefore, it can be described that it is difficult to realize a sufficiently small diameter of the puncture needle.

### SUMMARY

A puncture needle is disclosed that enables a liquid medicine to be efficiently injected even in a case where the puncture needle has a relatively small diameter and has high insertability into biological tissue.

The present disclosure can be achieved by any one of the following aspects (1) to (10).

(1) A puncture needle includes: a needle tube portion having a needle tube lumen extending in an axial direction; a cutting edge portion having a plurality of cutting surfaces formed at different positions around an axis and a plurality of ridge lines positioned at respective boundaries of the plurality of cutting surfaces, the cutting edge portion being arranged on a distal side of the needle tube portion; and one or more hole portions that are formed on a side surface of the needle tube portion and allow the needle tube lumen of the needle tube portion to communicate with an outside. The one or more hole portions are arranged on extension of the ridge lines in the axial direction, and two or more of the one or more hole portions are provided.

(2) In the puncture needle according to (1), on at least a proximal side of the one or more hole portions, an inner wall of the needle tube portion in which the one or more hole portions are formed intersects an external surface of the needle tube portion at an obtuse angle.

(3) In the puncture needle according to (1) or (2), each of the one or more hole portions has, in plan view, a substantially rectangular shape having a long side extending along the axial direction.

(4) In the puncture needle according to (3), a corner portion of each of the one or more hole portions is chamfered.

(5) In the puncture needle according to any one of (1) to (4), a groove portion extending from each of the one or more hole portions toward each of the ridge lines is formed in an external surface of the needle tube portion.

(6) In the puncture needle according to (5), the groove portion extends to at least a boundary position between the cutting edge portion and the needle tube portion.

(7) In the puncture needle according to any one of (1) to (6), the cutting edge portion has a solid structure and is fixed to the needle tube portion on the distal side of the needle tube portion.

(8) In the puncture needle according to any one of (1) to (7), the cutting edge portion has the ridge lines as many as the one or more hole portions.

According to the puncture needle described in (1), it is possible to provide a puncture needle that enables a liquid medicine to be efficiently injected even in a case where the puncture needle has a small diameter and has relatively high insertability into biological tissue.

(9) A puncture needle comprising: a needle tube portion having a needle tube lumen extending in an axial direction; a cutting edge portion having a plurality of cutting surfaces formed at different positions around an axis, the cutting edge portion being arranged on a distal side of the needle tube portion; and one or more hole portions that are formed on a side surface of the needle tube portion and allow the needle tube lumen of the needle tube portion to communicate with an outside of the needle tube portion, wherein the one or more hole portions are arranged on an extension of each of the plurality of plurality of cutting surfaces in the axial direction.

(10) A medical device that is retractably inserted into a delivery device, the medical device comprising: an outer sheath; and a puncture needle that is configured to be inserted into the outer sheath, the puncture needle including a needle tube portion having a needle tube lumen extending in an axial direction, a cutting edge portion, and one or more hole portions that are formed on a side surface of the needle tube portion and allow the needle tube lumen of the needle tube portion to communicate with an outside of the needle tube portion.

### BRIEF DESCRIPTION OF THE DRAWINGS

FIG. 1 is a perspective view of a medical device according to an embodiment.

FIG. 2 is a perspective view of the medical device according to the embodiment.

FIG. 3 is a partial cross-sectional view along an axial direction of the medical device according to the embodiment.

FIG. 4 is an enlarged partial cross-sectional view of a part of the medical device according to the embodiment.

FIG. 5 is an enlarged partial cross-sectional view of a part of the medical device according to the embodiment.

FIG. 6 is an axis-orthogonal cross-sectional view of the medical device taken along arrow 6A-6A in FIG. 3.

FIG. 7 is a perspective view illustrating the vicinity of a distal portion of a puncture device.

FIG. 8 is a perspective view illustrating the vicinity of the distal portion of the puncture device.

FIG. 9 is a perspective view illustrating the vicinity of the distal portion of the puncture device.

FIG. 10 is a perspective cross-sectional view along an axial direction of the distal portion of the puncture device.

FIG. 11 is a plan view of a puncture needle.

FIG. 12 is a front view of the puncture needle.

FIG. 13 is a cross-sectional view schematically illustrating an example of using the medical device according to the embodiment.

FIG. 14 is a cross-sectional view schematically illustrating the example of using the medical device according to the embodiment.

FIG. 15 is a cross-sectional view schematically illustrating the example of using the medical device according to the embodiment.

FIG. 16 is a cross-sectional view schematically illustrating the example of using the medical device according to the embodiment.

FIG. 17 is a cross-sectional view schematically illustrating the example of using the medical device according to the embodiment.

FIG. 18 is a perspective view for illustrating a medical device according to a modification example.

FIG. 19 is a perspective view for illustrating a lock mechanism additionally provided in the medical device according to the modification example.

### DETAILED DESCRIPTION

Set forth below with reference to the accompanying drawings is a detailed description of embodiments of a puncture needle. Dimensional ratios in the drawings are exaggerated for convenience of description and may be different from actual ratios.

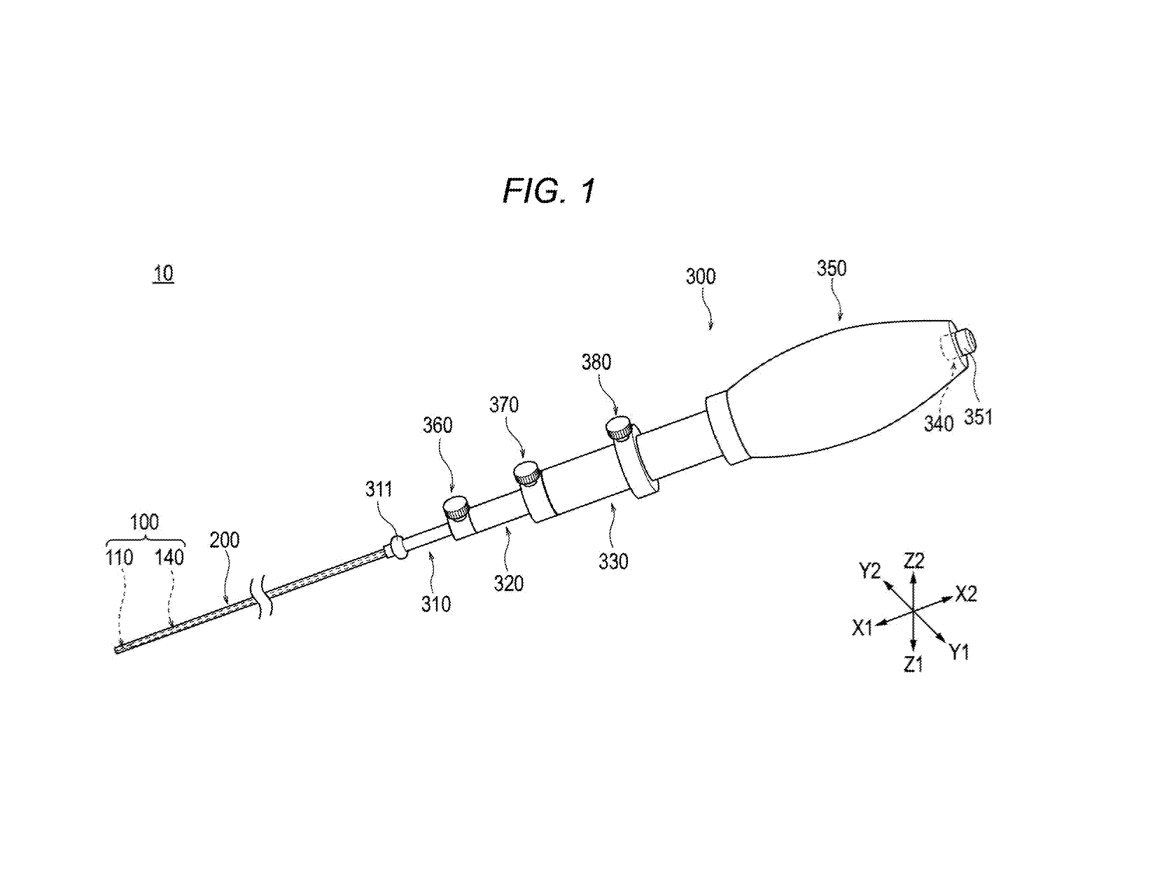

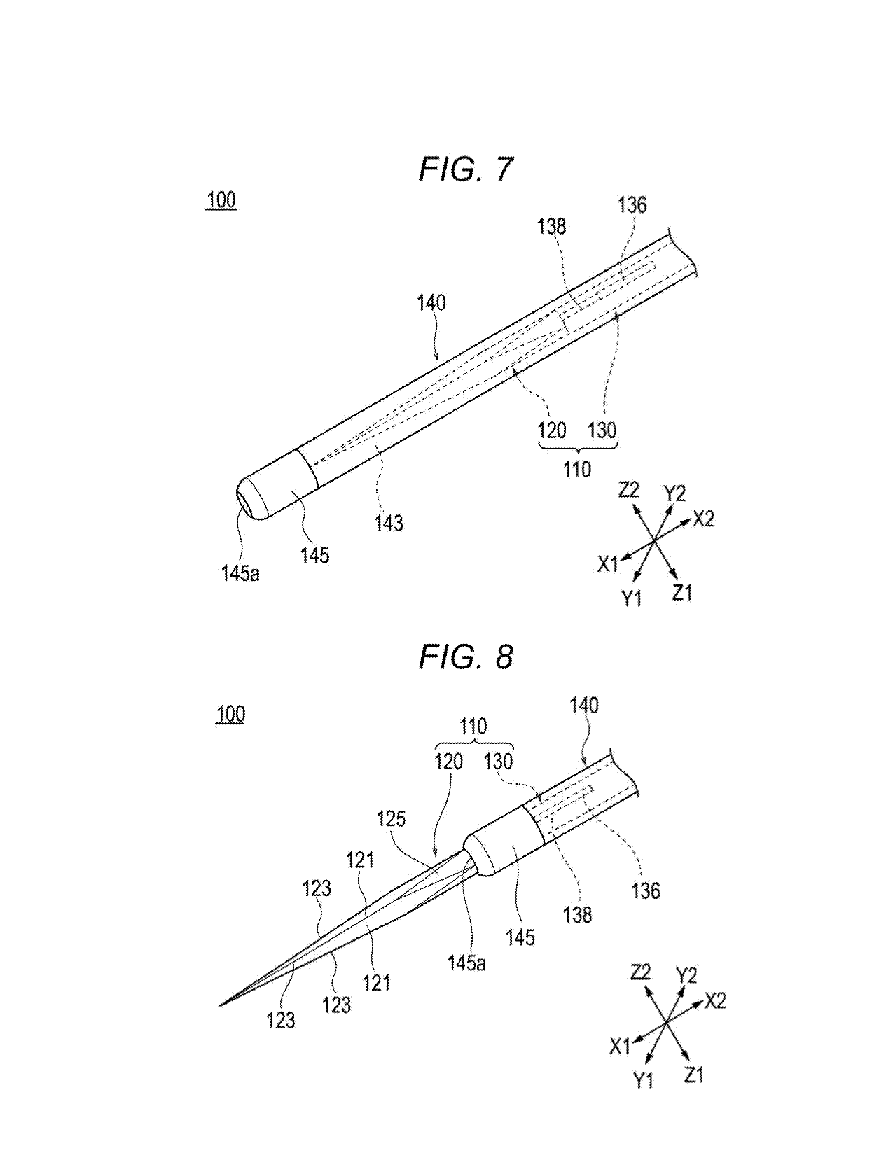

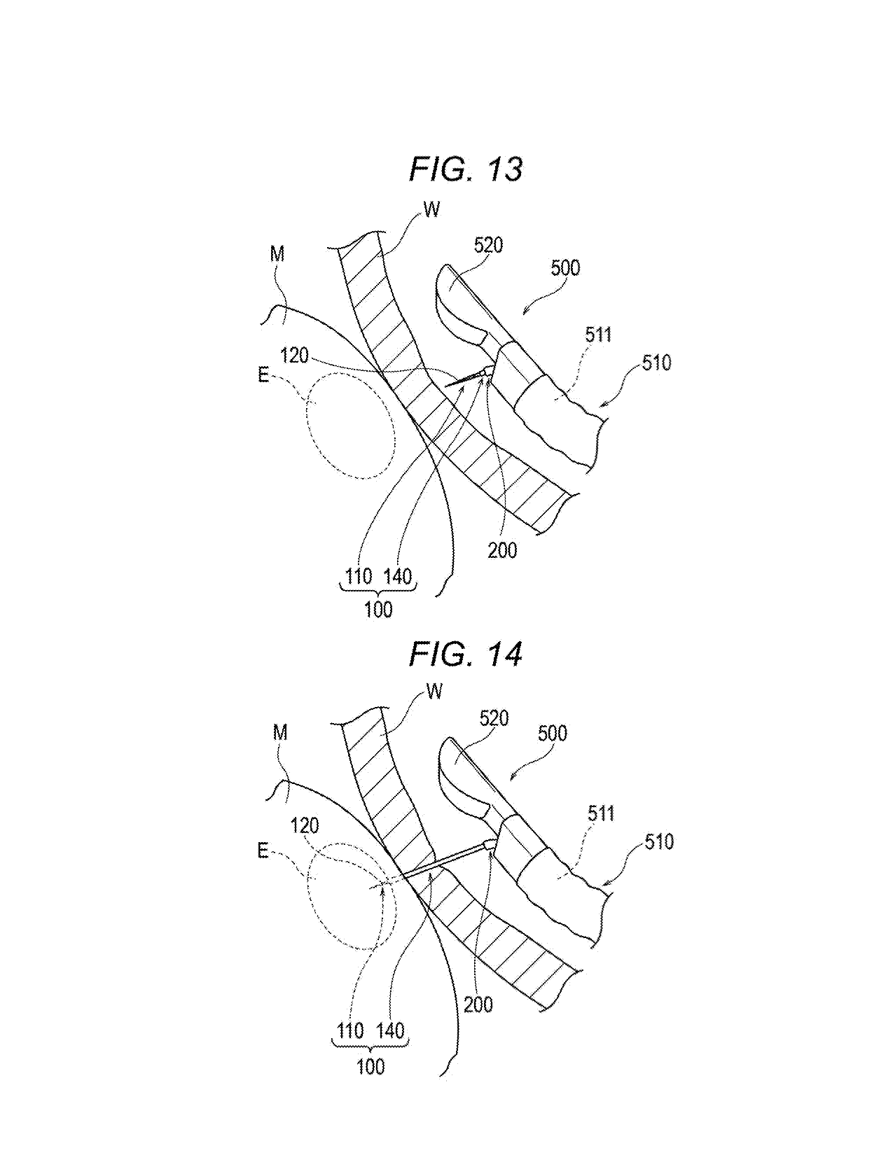

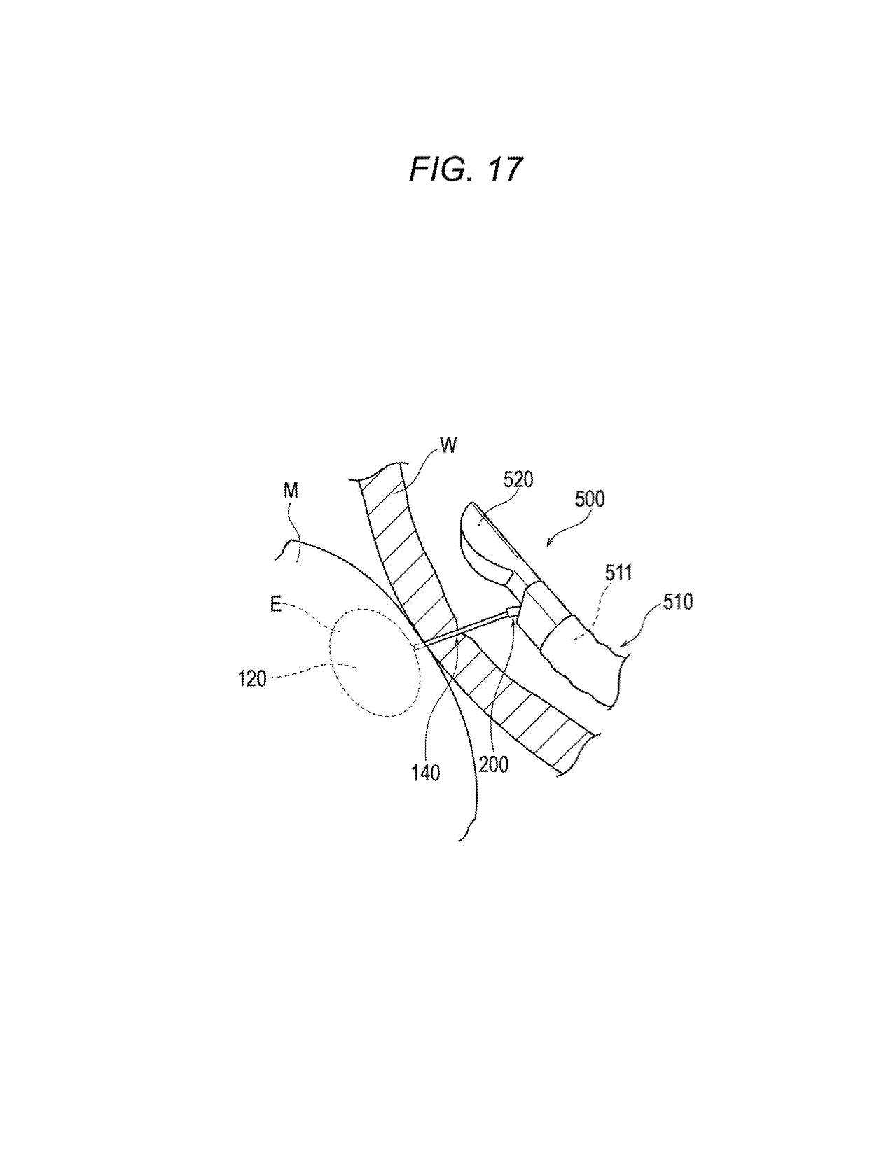

FIGS. 1 to 6 are views illustrating an overall configuration of a medical device 10 according to an embodiment, FIGS. 7 to 12 are views for illustrating a puncture device 100 according to the embodiment, and FIGS. 13 to 17 are views for illustrating a procedure using the medical device 10.

In the present specification, each direction regarding the medical device 10 is defined as follows. A side of the medical device 10 on which the medical device is introduced into a living body (a side denoted by an arrow X1 in the drawings) is defined as a distal side, and an end portion on the distal side in each member and each component is defined as a “distal portion”. In addition, a side on which a grip portion 350 of a handheld handle portion 300 of the medical device 10 is arranged (a side denoted by an arrow X2 in the drawings) is defined as a proximal side, and an end portion positioned on the proximal side in each member and each component is defined as a “proximal portion”. In addition, a direction from the distal portion toward the proximal portion (or from the proximal portion toward the distal portion) is defined as an “axial direction (a direction denoted by the arrows X1-X2)”. Arrows Y1-Y2 in each drawing denote a width direction orthogonal to the axial direction, and arrows Z1-Z2 denote a height direction orthogonal to both the axial direction and the width direction.

### Medical Device 10

As illustrated in FIGS. 13 to 17, the medical device 10 in which a puncture needle 110 according to the present embodiment is installed is used by being retractably inserted into a delivery device.

In the present embodiment, a known endoscopic device 500 in the medical field is exemplified as the delivery device. When the medical device 10 is delivered to a target site E in the living body, the medical device can be retractably inserted into a channel 511 of an insertion portion 510 included in the endoscopic device 500. The endoscopic device 500 can be, for example, a known ultrasound endoscope in which an ultrasound image pickup unit 520 is arranged at a distal portion of the insertion portion 510. However, a specific structure, product specifications, a product type, or the like of the endoscopic device 500 are not particularly limited.

As long as the delivery device has a passage (a lumen) into which the medical device 10 is retractably inserted, the product specifications and the specific structure of the delivery device are not particularly limited. For example, the delivery device may be a catheter or the like having a lumen into which the medical device 10 is retractably inserted.

As illustrated in FIGS. 1 to 3, the medical device 10 includes the puncture device 100 having the puncture needle 110, an outer sheath 200 allowing the puncture device 100 to penetrate (extend through the outer sheath 200), and the handheld handle portion 300 arranged on a proximal side of the puncture device 100 and the outer sheath 200.

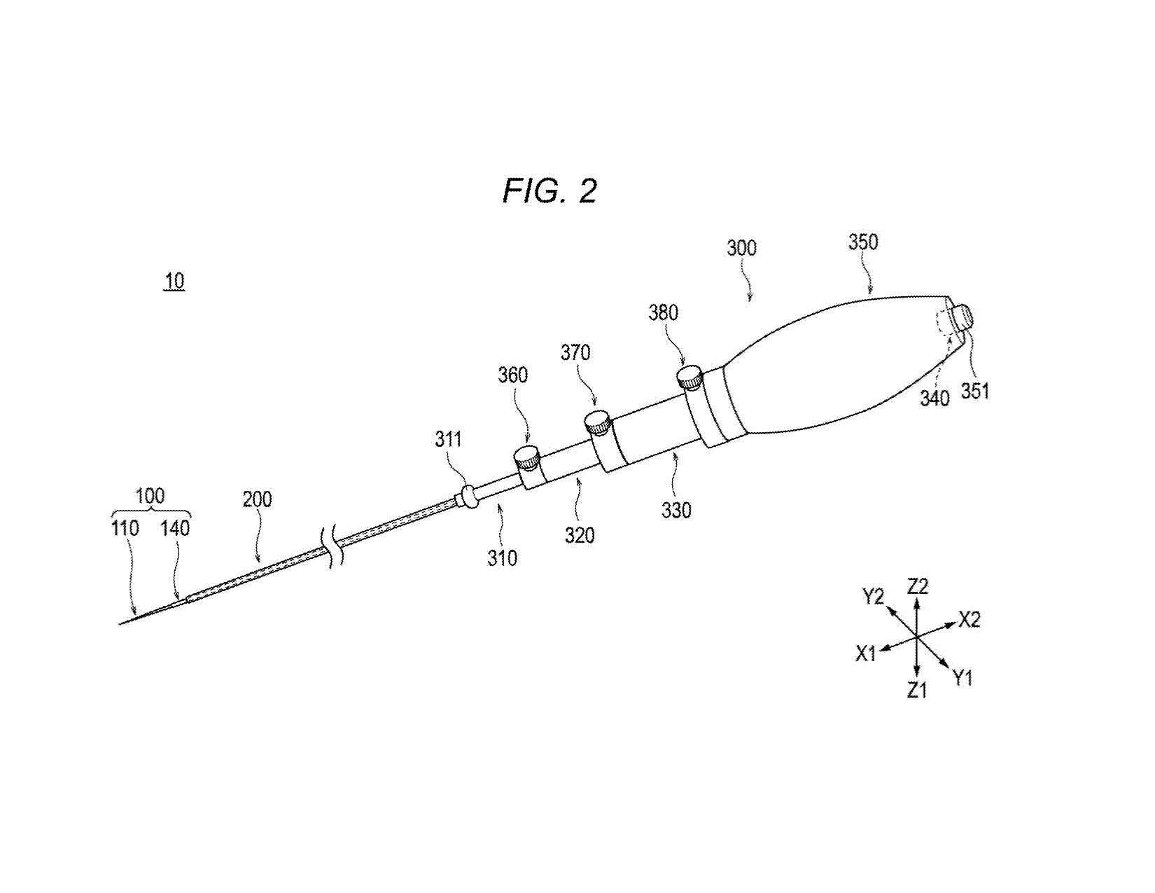

In the medical device 10, the puncture device 100, the inner sheath 140, and the outer sheath 200 are coupled to each portion of the handheld handle portion 300, and relative positions of the above-described three constituent members in the axial direction can be changed and adjusted by manipulating the handheld handle portion 300. FIG. 1 illustrates a state in which the inner sheath 140 and the puncture device 100 are accommodated in the outer sheath 200, and FIG. 2 illustrates an aspect when the inner sheath 140 and the puncture device 100 project toward a distal side of the outer sheath 200.

### Outer Sheath 200

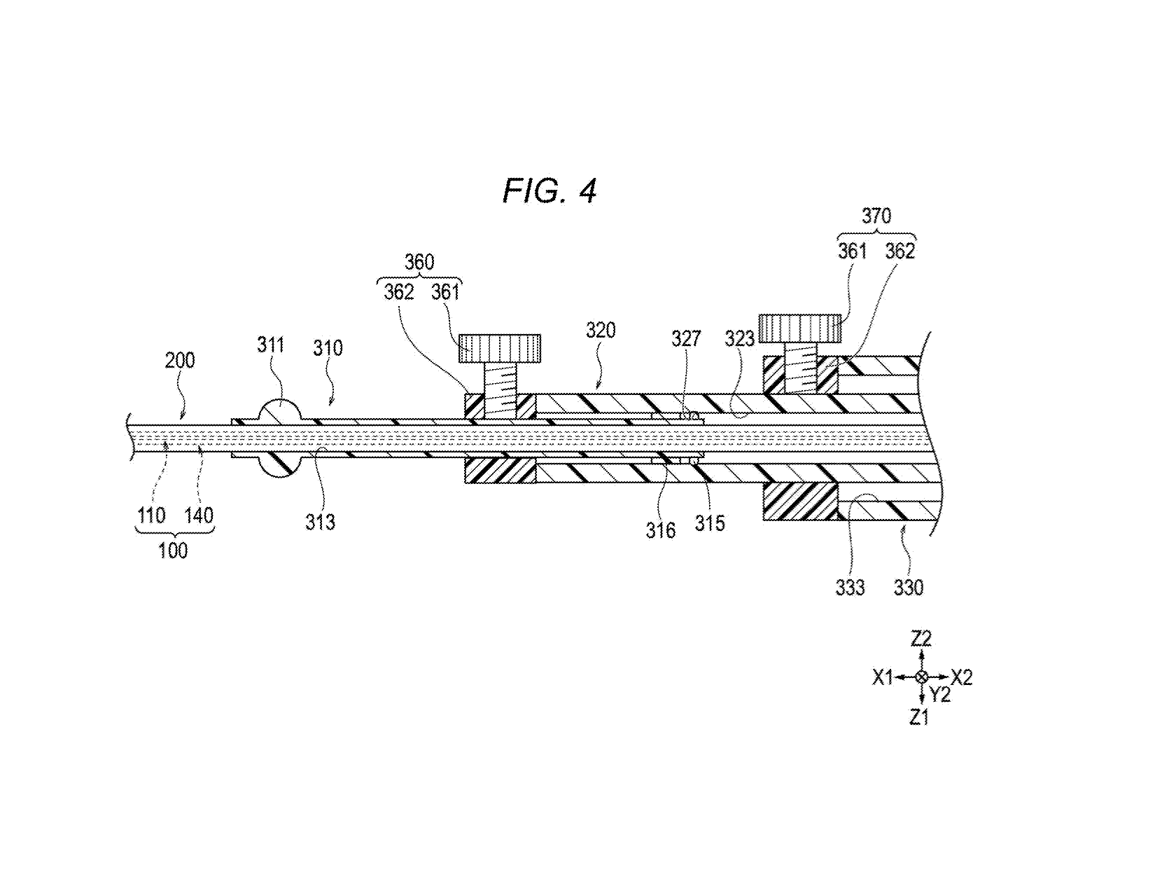

As illustrated in FIGS. 4-6, the outer sheath 200 is configured of a member with a multi-tube structure which has predetermined lumens 203 and 204 in the outer sheath 200.

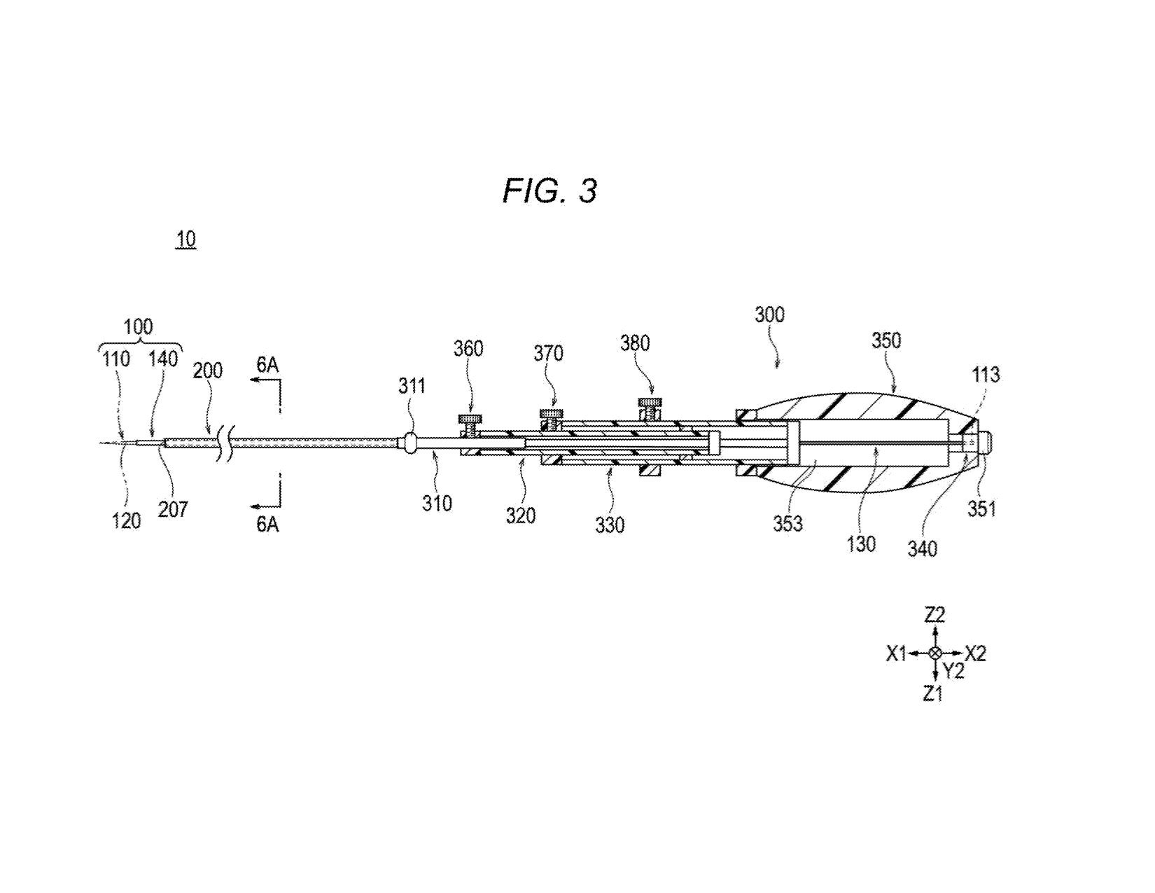

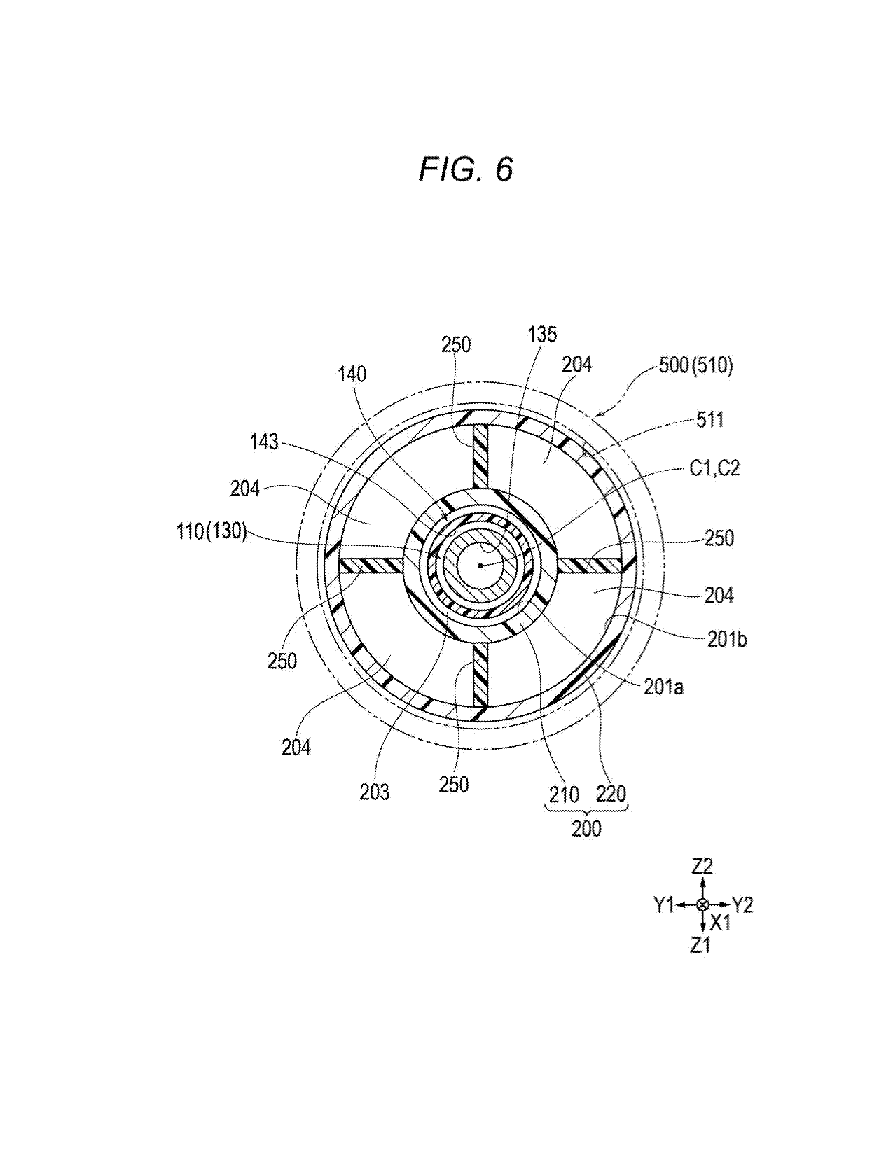

As illustrated in FIG. 6, the outer sheath 200 has a first surface 201a defining, on an inner side of first surface 201a, the first lumen 203 enabling the puncture device 100 to penetrate (or extend through), and a second surface 201b defining at least one second lumen 204 between the first surface 201a and the second surface 201b. FIG. 6 is an axis-orthogonal cross-sectional view taken along arrow 6A-6A in FIG. 3.

The outer sheath 200 includes an inner tube 210 having the first surface 201a and the first lumen 203, an outer tube 220 having the second surface 201b and the second lumen 204, and a fixing member 250 that is arranged between the inner tube 210 and the outer tube 220 and fixes the inner tube 210 and the outer tube 220.

The first surface 201a is configured of an inner surface (an internal surface) of the inner tube 210. The second surface 201b is configured of an inner surface (an internal surface) of the outer tube 220. The first lumen 203 is formed inside the inner tube 210 and extends over substantially the entire length of the inner tube 210 in the axial direction. The second lumen 204 is demarcated between an outer surface of the inner tube 210 and the inner surface of the outer tube 220 and extends over substantially the entire length of the inner tube 210 and the outer tube 220 in the axial direction.

As illustrated in FIG. 6, in the present embodiment, four second lumens 204 are formed at equal intervals (axisymmetrically) in a circumferential direction in the outer tube 220. In addition, the second lumens 204 are individually partitioned by the fixing member 250 to have substantially the same cross-sectional area at different positions around central axes C1 and C2 of the inner tube 210 and the outer tube 220 in the circumferential direction. Hence, the second lumen 204 is demarcated by the outer surface of the inner tube 210, the inner surface of the outer tube 220, and exposed surfaces of the fixing members 250. The number of the second lumens 204 is not particularly limited.

The fixing member 250 fixes the inner tube 210 and the outer tube 220, thereby maintaining a relative positional relationship between the inner tube 210 and the outer tube 220 (a positional relationship in the axis-orthogonal cross-sectional view illustrated in FIG. 6).

In the present embodiment, as illustrated in FIG. 6, the inner tube 210 and the outer tube 220 are concentrically arranged by the fixing member 250. In other words, in the axis-orthogonal cross-sectional view illustrated in FIG. 6, the central axis C1 of the inner tube 210 and the central axis C2 of the outer tube 220 are arranged at an overlap position.

As illustrated in FIG. 6, four fixing members 250 are arranged at equal intervals in a circumferential direction of the outer sheath 200, around a central axis of the outer sheath 200 (at the same position as the central axes C1 and C2). An angle difference between the fixing members 250 in the circumferential direction is approximately 90°.

The number, the arrangement, the cross-sectional shape (the cross-sectional shape illustrated in FIG. 6), or the like of the fixing members 250 are not particularly limited as long as the fixing members exhibit a function of fixing the inner tube 210 and the outer tube 220. For example, the fixing member 250 can also be configured of a single member that extends helically around the central axis of the outer sheath 200.

As illustrated in FIG. 3, a distal opening portion 207 that enables the puncture device 100 to project from the outer sheath 200 is provided at the distal end of the outer sheath 200.

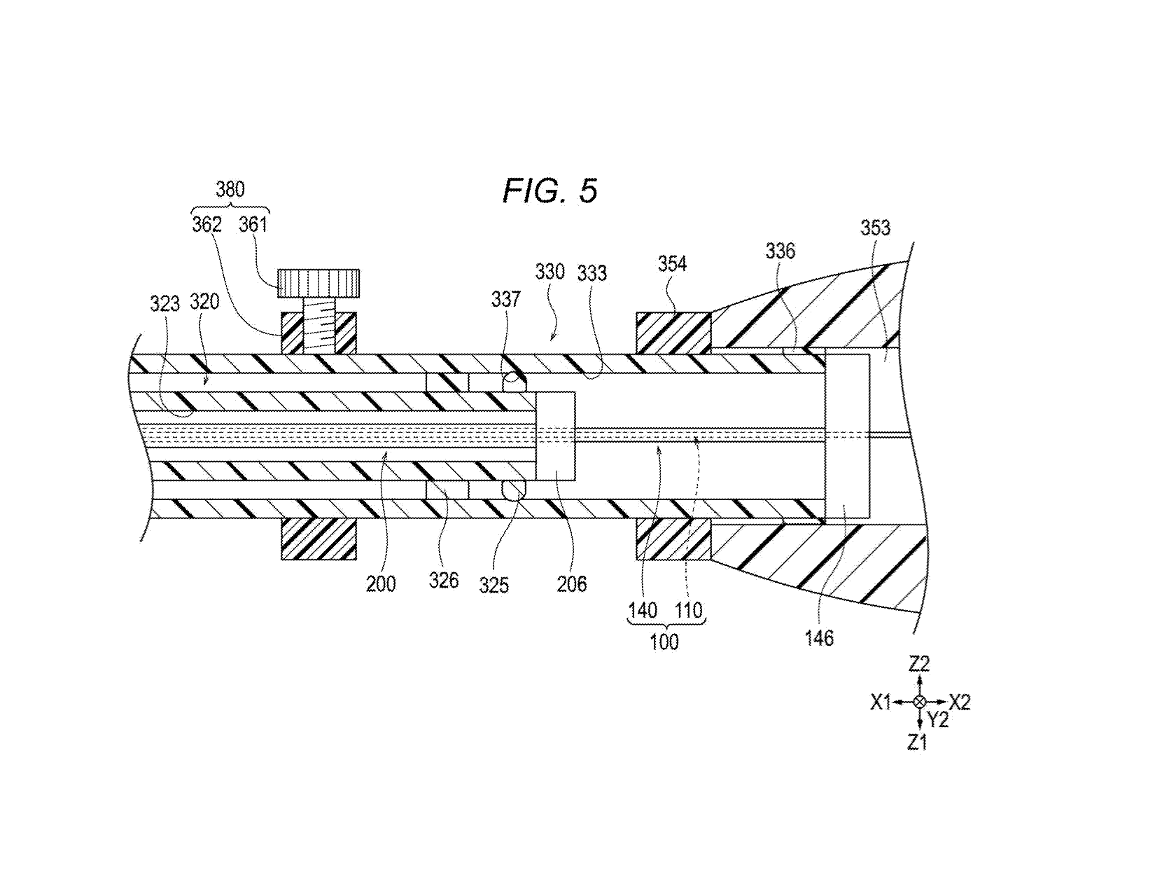

As illustrated in FIG. 5, a proximal portion 206 of the outer sheath 200 is coupled to a first coupling portion 320 to be described below. Therefore, when the first coupling portion 320 is advanced and retracted in the axial direction, the outer sheath 200 is advanced and retracted in the axial direction in conjunction with the advancing and retracting movement of the first coupling portion 320. The proximal portion of the outer sheath 200 means a proximal portion of the inner tube 210 and a proximal portion of the outer tube 220.

The outer sheath 200 (the inner tube 210 and the outer tube 220) and the inner sheath 140 to be described below can be made of, for example, a material similar to a material used as a constituent material of a known catheter or the like in the medical field. Examples of the material of the outer sheath 200 and the inner sheath 140 can include a polyolefin such as polyethylene, polypropylene, an ethylene-propylene copolymer, or an ethylene-vinyl acetate copolymer, a thermoplastic resin such as soft polyvinyl chloride, various types of elastomer such as a polyurethane elastomer, a polyamide elastomer, or a polyester elastomer, or a crystalline plastic such as a polyamide, crystalline polyethylene, and crystalline polypropylene.

The fixing member 250 is preferably made of a relatively flexible resin material so as not to excessively increase the stiffness of the outer sheath 200. Examples of the material of the fixing member 250 can include a polyolefin (for example, polyethylene, polypropylene, polybutene, an ethylene-propylene copolymer, an ethylene-vinyl acetate copolymer, ionomer, or a mixture of two or more types of the above-mentioned polyolefins), polyvinyl chloride, polyamide, polyamide elastomer, polyurethane, polyurethane elastomer, and polyimide.

### Puncture Device 100

As illustrated in FIGS. 3 and 7 to 9, the puncture device 100 includes the puncture needle 110 having a cutting edge portion 120 and a needle tube portion 130, and the inner sheath 140 having a third lumen 143 into which the puncture needle 110 is retractably inserted.

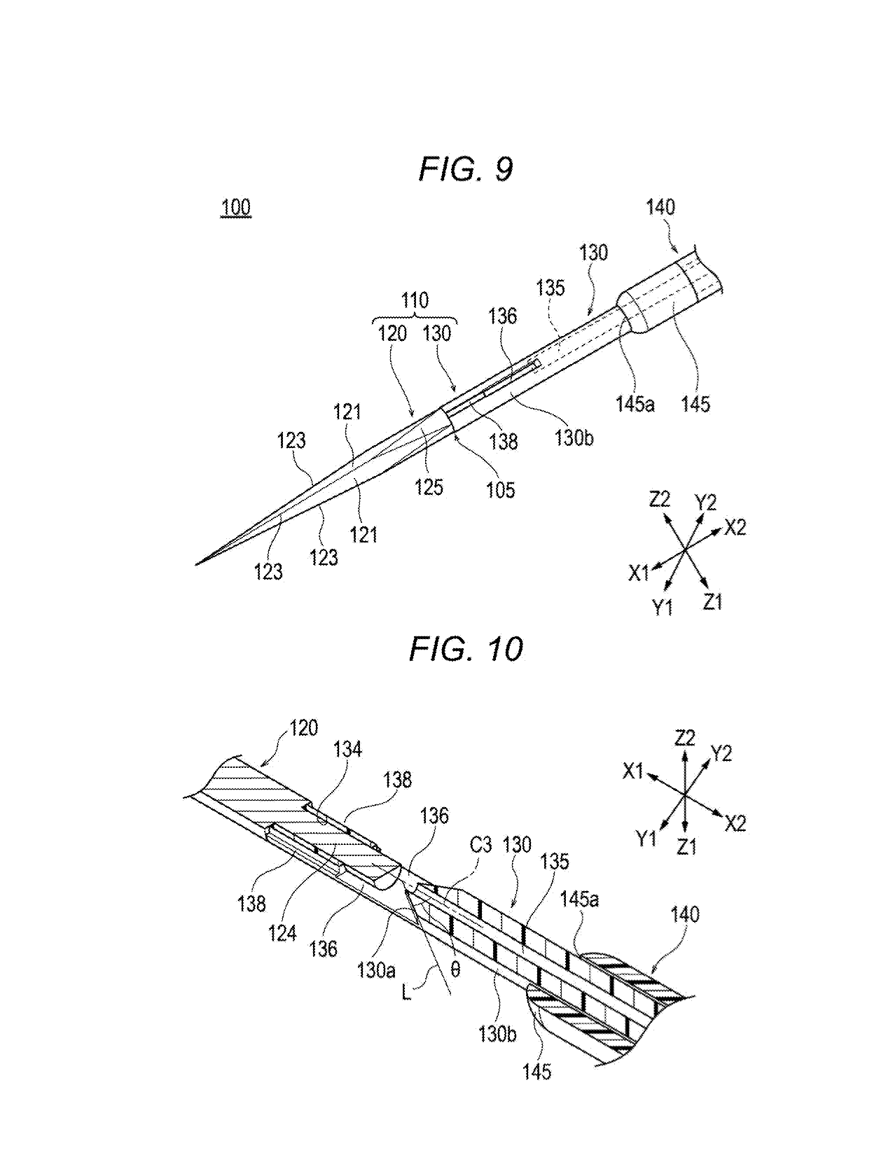

FIG. 7 illustrates a state in which the cutting edge portion 120 and the needle tube portion 130 are accommodated in the third lumen 143 of the inner sheath 140. FIG. 8 illustrates an aspect when the cutting edge portion 120 projects from the third lumen 143 of the inner sheath 140. FIG. 9 illustrates an aspect when the cutting edge portion 120 and the vicinity of a distal portion of the needle tube portion 130 project from the third lumen 143 of the inner sheath 140.

### Inner Sheath 140

As illustrated in FIGS. 8-10, a distal member 145 is attached to a distal portion of the inner sheath 140. The distal member 145 can be made of, for example, a metal or the like to which X-ray contrast properties are imparted. Alternatively, the distal member 145 may have a groove, a recess, or a protrusion is provided to have high ultrasonic reflectivity. In a case where the distal member 145 is made of metal or the like to which the X-ray contrast properties are imparted, a distal position of the inner sheath 140 (a position of the distal member 145) can be visually recognized and located under fluoroscopic control. In addition, in a case where the distal member 145 is configured to have the high ultrasonic reflectivity, the distal position of the inner sheath 140 (the position of the distal member 145) can be visually recognized and located by an ultrasound device. A similar effect can be obtained by providing a radiopaque marker or an ultrasound marker on the distal member 145. A distal opening portion 145a that enables the puncture needle 110 to protrude is provided at a distal end of the distal member 145.

As illustrated in FIG. 5, a proximal portion 146 of the inner sheath 140 is coupled to the handheld handle portion 300 at a second coupling portion 330 to be described below. Therefore, when the second coupling portion 330 is advanced and retracted in the axial direction, the inner sheath 140 is advanced and retracted in the axial direction in conjunction with the advancing and retracting movement of the second coupling portion 330. Here, the proximal portion 146 of the inner sheath 140 may include a tubular member capable of coupling the inner sheath 140 in the lumen, and a cylindrical member which allows the tubular member to penetrate (or extend through) and to which the tubular member is fixed.

As illustrated in FIG. 5, the proximal portion 146 of the inner sheath 140 coupled to the second coupling portion 330 is arranged on the proximal side with respect to the proximal portion 206 of the outer sheath 200 coupled to the first coupling portion 320. The inner sheath 140 is inserted into the first lumen 203 (see FIG. 6) from a position on the proximal side with respect to the proximal portion 206 of the outer sheath 200.

As illustrated in FIG. 3, a proximal portion 113 of the puncture needle 110 (a proximal portion of the needle tube portion 130) is coupled to a third coupling portion 340 to be described below. In addition, the third coupling portion 340 is coupled to the grip portion 350 to be described below. Therefore, when the third coupling portion 340 is advanced and retracted in the axial direction together with the grip portion 350, the puncture needle 110 is advanced and retracted in the axial direction in conjunction with the advancing and retracting movement of the third coupling portion 340.

As illustrated in FIGS. 3 and 5, the proximal portion 113 of the puncture needle 110 coupled to the third coupling portion 340 is arranged on the proximal side with respect to the proximal portion 146 of the inner sheath 140 coupled to the second coupling portion 330. The puncture needle 110 is inserted into the third lumen 143 (see FIG. 6) from a position on the proximal side with respect to the proximal portion 146 of the inner sheath 140.

### Puncture Needle 110

As illustrated in FIGS. 7-9, the puncture needle 110 includes the needle tube portion 130 and the cutting edge portion 120.

The needle tube portion 130 includes a needle tube lumen 135 extending in the axial direction. An outer diameter of the needle tube portion 130 is not particularly limited, and is preferably 0.51 mm or smaller, and more preferably 0.41 mm or smaller, from the viewpoint of reducing the generation of an unintended perforation at the time of puncture.

The cutting edge portion 120 is arranged on a distal side of the needle tube portion 130.

The cutting edge portion 120 has a plurality of cutting surfaces 121 formed at different positions around an axis and a plurality of ridge lines 123 positioned at respective boundaries of the plurality of cutting surfaces 121.

The puncture needle 110 has one or more hole portions 136 that are formed in a side surface of the needle tube portion 130 and allow the needle tube lumen 135 of the needle tube portion 130 to communicate with the outside of the needle tube portion 130. The one or more hole portions 136 are arranged on extension of the ridge lines 123 in the axial direction. In addition, preferably two or more hole portions 136 are provided in the needle tube portion 130.

As illustrated in FIG. 12, the cutting edge portion 120 can be configured to have, for example, four cutting surfaces 121 and four ridge lines 123 positioned at boundaries between the individual cutting surfaces 121. The individual ridge lines 123 are arranged at equal intervals of approximately 90° at different positions in the circumferential direction around a central axis C3 of the puncture needle 110.

As illustrated in FIG. 12, each of the four cutting surfaces 121 included in the cutting edge portion 120 is configured of a tapered surface extending in a substantially straight line shape to be gradually separated from the central axis C3 of the puncture needle 110, from a distal end toward a proximal side (the needle tube portion 130 side) of the puncture needle 110.

As illustrated in FIG. 12, the needle tube portion 130 can be configured to have a number of the one or more hole portions 136, which are equal (i.e., as many as) to the number of ridge lines 123. In the present embodiment, four hole portions 136 equal to the number of ridge lines 123 are provided in the side surface of the needle tube portion 130.

The one or more hole portions 136 can be arranged at positions corresponding to the ridge lines 123 in the circumferential direction around the central axis C3 of the puncture needle 110. In the present embodiment, the one or more hole portions 136 are arranged at different positions in the circumferential direction around the central axis C3 of the puncture needle 110 with an equal angle difference of approximately 90°.

As illustrated in FIG. 10, the cutting edge portion 120 has a solid structure. In other words, the needle tube lumen 135 is not formed inside the cutting edge portion 120.

As illustrated in FIG. 10, the cutting edge portion 120 is fixed to the needle tube portion 130 on the distal side of the needle tube portion 130.

In the puncture needle 110, a portion constituting the cutting edge portion 120 and a portion constituting the needle tube portion 130 are configured of respective separate members. In other words, the puncture needle 110 includes two members.

The cutting edge portion 120 has a first fixing portion 124 provided at a proximal portion of the cutting edge portion 120. The needle tube portion 130 has a second fixing portion 134 provided at a distal portion of the needle tube portion 130. The second fixing portion 134 is arranged on the distal side with respect to the one or more hole portions 136.

The second fixing portion 134 has a recess-shaped space in which the first fixing portion 124 can be fitted. The cutting edge portion 120 is coupled to the needle tube portion 130 by fitting the first fixing portion 124 in the second fixing portion 134. The coupling between the first fixing portion 124 and the second fixing portion 134 can be reinforced by an adhesive, welding, or the like, as appropriate.

As illustrated in FIG. 3, the puncture needle 110 has a proximal opening portion formed in the proximal portion 113. The proximal opening portion of the puncture needle 110 is arranged to communicate with a first port 351 integrally coupled to the third coupling portion 340. When a predetermined liquid medicine supply instrument (for example, a known syringe pump in the medical field) is coupled to the first port 351 and a liquid medicine is supplied, the liquid medicine flows into the needle tube lumen 135 via the first port 351 and the proximal opening portion of the puncture needle 110. The inflow of the liquid medicine into the needle tube lumen 135 may be performed via the one or more hole portions 136 to be described below. That is, an instrument capable of suctioning may be coupled to the first port 351, and the instrument may perform suctioning in a state in which the one or more hole portions 136 project from the inner sheath 140 (a state in which the one or more hole portions 136 are not covered by the inner sheath 140), so that the liquid medicine may flow into the needle tube lumen 135 via the one or more hole portions 136.

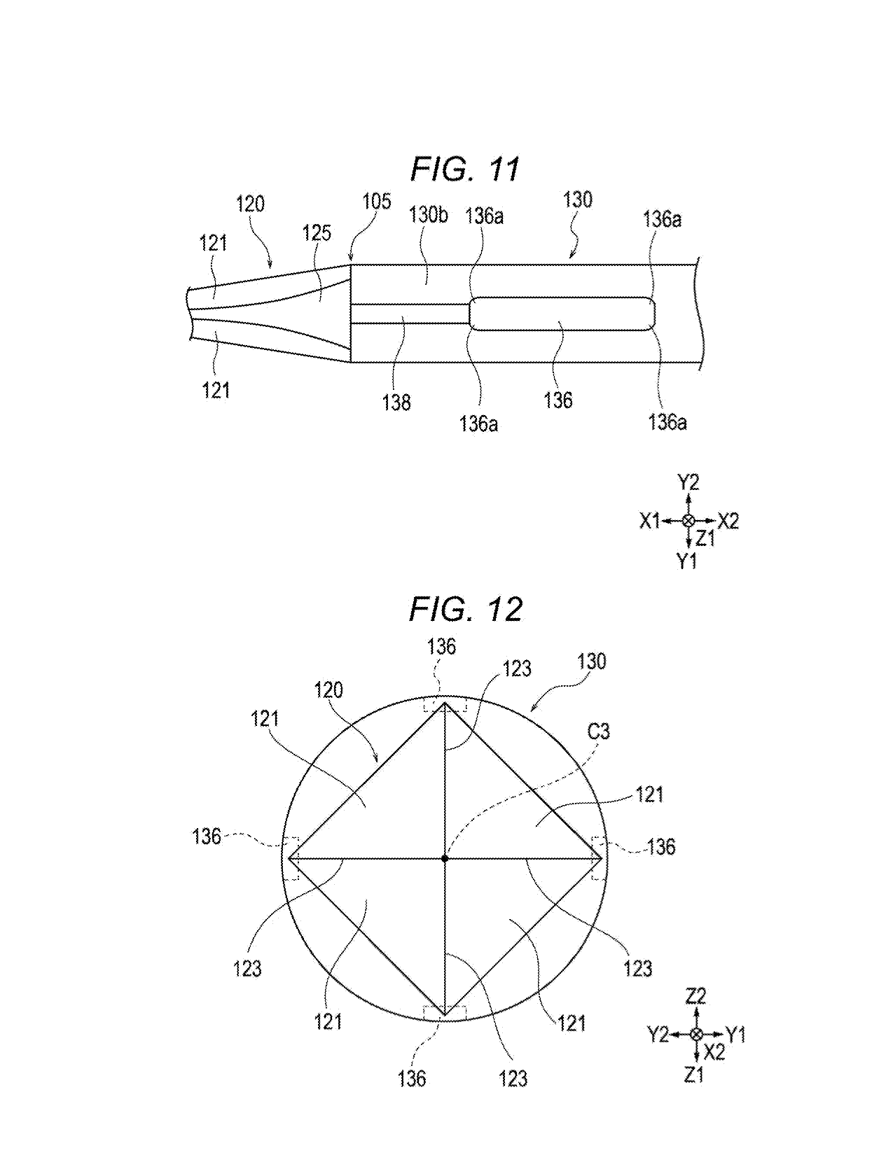

As illustrated in FIGS. 10 and 11, the needle tube lumen 135 is connected to the one or more hole portions 136 formed in the vicinity of the distal portion of the needle tube portion 130. When the liquid medicine flowing into the needle tube lumen 135 moves to the distal side in the axial direction along the needle tube lumen 135 and then, reaches the vicinity of the distal portion of the needle tube portion 130, the liquid medicine is released to the outside of the needle tube portion 130 (the outside of the puncture needle 110) via the one or more hole portions 136. The puncture device 100 is retractably inserted into the third lumen 143 of the inner sheath 140, thereby enabling the liquid medicine to be prevented from leaking from the one or more hole portions 136 of the puncture device 100 in a state in which the cutting edge portion 120 and the needle tube portion 130 are accommodated in the third lumen 143 of the inner sheath 140.

In a procedure using the medical device 10, as illustrated in FIG. 9, the liquid medicine can be locally administered to the target site E (see FIG. 16) via the one or more hole portions 136 by supplying the liquid medicine into the needle tube lumen 135 in a state in which the one or more hole portions 136 formed in the needle tube portion 130 project from the inner sheath 140 (a state in which the one or more hole portions 136 are not covered by the inner sheath 140).

As illustrated in FIG. 10, at least on a proximal side of the hole portion 136, an inner wall 130a of the needle tube portion 130 having the one or more hole portions 136 formed on an inner side of the inner wall 130a of the needle tube portion 130 intersects an external surface 130b of the needle tube portion 130 at an obtuse angle.

That “the inner wall 130a of the needle tube portion 130 intersects the external surface 130b of the needle tube portion 130 at an obtuse angle” described above means that an angle θ formed between a virtual straight line L parallel to the inner wall 130a of the needle tube portion 130 and the central axis C3 of the puncture needle 110 is an acute angle as illustrated in FIG. 10. The angle θ is not particularly limited as long as the angle is an acute angle, and is preferably, for example, from 45° to 60°.

The hole portion 136 can be configured to have a substantially rectangular shape having a long side extending along the axial direction, in a plan view illustrated in FIG. 11.

As illustrated in FIG. 11, corner portions 136a (corner portions positioned at four corners) of the hole portion 136 can be chamfered. In other words, the individual corner portions 136a of the hole portion 136 formed in the substantially rectangular shape can be formed in a rounded shape in a plan view illustrated in FIG. 11.

As illustrated in FIGS. 9 and 11, a groove portion 138 extending coaxially with the ridge line 123 from a distal side end portion of the hole portion 136 toward the ridge line 123 is formed in the external surface 130b of the needle tube portion 130. A radiopaque marker or an ultrasound marker may be provided on the external surface 130b of the needle tube portion 130 around the groove portion 138. Here, the radiopaque marker is formed by making, of a metal or the like to which X-ray contrast properties are imparted, the external surface 130b of the needle tube portion 130 around the groove portion 138, or providing, on the external surface 130b of the needle tube portion 130, a metal or the like to which X-ray contrast properties are imparted. The ultrasound marker is formed by providing an inclination at an edge portion of the groove portion 138 or providing another groove, a recess, or a protrusion around the groove portion 138. By providing the above-described marker, a position of the hole portion 136 can be visually recognized and located under fluoroscopic control (with a CT device) or with an ultrasound device.

The puncture needle 110 has four groove portions 138 connected to the four respective hole portions 136 arranged to correspond to the four respective ridge lines 123 arranged at different positions in the circumferential direction around the central axis C3 of the puncture needle 110.

As illustrated in FIG. 11, a distal side end portion of the groove portion 138 can be provided to extend to at least a boundary position 105 between the cutting edge portion 120 and the needle tube portion 130.

The above-described “boundary position 105” is a boundary position between a proximal end of the cutting edge portion 120 and the distal end of the needle tube portion 130.

For example, the groove portion 138 may extend further toward the cutting edge portion 120 (the distal side) with respect to the boundary position 105. Specifically, in the plan view illustrated in FIG. 11, the groove portion 138 can extend to any position of a proximal portion 125 positioned in the vicinity of the proximal portion of the cutting edge portion 120. By providing the groove portion 138 as described above, the liquid medicine discharged from the hole portion 136 is more smoothly guided to the ridge line 123 via the groove portion 138. The proximal portion 125 is positioned on the proximal side with respect to a proximal end of the ridge line 123 and forms a portion at which the ridge line 123 is not formed.

The groove portion 138 extends in a substantially straight line shape from the hole portion 136 toward the ridge line 123 in a plan view illustrated in FIG. 11. The groove portion 138 is configured to have substantially the same width (a length dimension in a direction orthogonal to the axial direction) along the axial direction. In addition, the groove portion 138 is continuously connected from the hole portion 136 to the boundary position 105 without interruption.

For example, the groove portion 138 may change in width midway from the hole portion 136 toward the ridge line 123 or may extend to meander. In addition, the groove portion 138 may be configured to have a plurality of portions that are not continuously connected in the axial direction but are provided intermittently at intervals. However, as illustrated in FIG. 11, in a case where the groove portion 138 is configured to extend in a substantially straight line shape with the same width from the hole portion 136 toward the ridge line 123 and be continuously connected from the hole portion 136 toward the ridge line 123 without interruption, the liquid medicine discharged from the hole portion 136 can be more efficiently guided to the ridge line 123.

The puncture needle 110 (the cutting edge portion 120 and the needle tube portion 130) can be configured of, for example, a metal needle including a metal as a constituent material. As the metal contained in the puncture needle 110, for example, stainless steel, aluminum, an aluminum alloy, titanium, a titanium alloy, or other metals can be used.

A specific structure (for example, the number and shape of the cutting surfaces 121, the number and shape of the ridge lines 123, or the like) of the cutting edge portion 120 is not particularly limited as long as the cutting edge portion 120 has the plurality of ridge lines 123 and the plurality of cutting surfaces 121. For example, the cutting edge portion 120 may have a hollow structure in which a lumen for allowing the liquid medicine to flow into the cutting edge portion 120 is formed or may have a distal opening portion that enables the liquid medicine to be discharged from a distal end of the cutting edge portion 120.

In addition, the cutting edge portion 120 and the needle tube portion 130 can be configured of a single integrated member.

In addition, the needle tube portion 130 may not have the groove portions 138.

In addition, the shape of the hole portion 136 in plan view is not particularly limited and may be, for example, a circular shape, an elliptical shape, a square shape, a rhombus shape, or the like.

### Handheld Handle Portion 300

As illustrated in FIGS. 3-5, the handheld handle portion 300 includes a first coupling portion 320 coupled to the outer sheath 200 and a second coupling portion 330 which is coupled to at least a part of the puncture device 100 and is relatively advanced and retracted with respect to the first coupling portion 320 so that a distal end of the puncture device 100 protrudes from the distal end of the outer sheath 200.

As illustrated in FIGS. 3 and 4, a distal side coupling portion 310 is coupled to a distal portion of the first coupling portion 320.

The distal side coupling portion 310 has a coupling target portion 311 arranged in the vicinity of the distal portion. The coupling target portion 311 is configured in a couplable manner to the endoscopic device 500. The coupling target portion 311 can be configured of, for example, a known Luer lock connector capable of performing locking/unlocking with respect to a device insertion port included in the endoscopic device 500.

The distal side coupling portion 310 includes a lumen 313 allowing the outer sheath 200 and the puncture device 100 to penetrate (or extend through) the distal side coupling portion 310.

As illustrated in FIGS. 3 and 4, a first stopper 360 is arranged on the distal side of the first coupling portion 320. The first stopper 360 has a configuration in which the first stopper is movable along an outer surface of the distal side coupling portion 310.

The first stopper 360 includes a fastener 361 for fixing the first stopper 360 to the distal side coupling portion 310, and a support portion 362 for supporting the fastener 361.

The fastener 361 can be configured of, for example, a screw having a male thread portion. The support portion 362 can be configured to have, for example, a female thread portion to which the fastener 361 can be screwed.

An operator who manipulates the medical device 10 can fix the first stopper 360 at an arbitrary position of the distal side coupling portion 310 in the axial direction by screwing the fastener 361 in the support portion 362 and bringing the fastener 361 into contact with the outer surface of the distal side coupling portion 310. In addition, the operator can move the first stopper 360 along the outer surface of the distal side coupling portion 310 by manipulating the fastener 361 to weaken a fixing force of the first stopper 360 with respect to the distal side coupling portion 310.

The first coupling portion 320 can be advanced and retracted in the axial direction along the distal side coupling portion 310. A maximum advance position (an advance limit position) of the first coupling portion 320 in the axial direction is set by a position at which the first stopper 360 is arranged. In other words, when the operator moves the first coupling portion 320 in the axial direction, the operator can advance the first coupling portion 320 to a position at which the distal portion of the first coupling portion 320 is brought into contact with the first stopper 360. In addition, an exposure length by which the distal side coupling portion 310 is exposed from the first coupling portion 320 is adjusted by a position of the first stopper 360.

A specific configuration of the first stopper 360 is not particularly limited as long as the maximum advance position of the first coupling portion 320 can be set as described above. The same applies to a second stopper 370 and a third stopper 380 to be described below. In the present embodiment, the second stopper 370 and the third stopper 380 have substantially the same configuration as that of the first stopper 360. Therefore, in the following description, the descriptions of the configurations of the second stopper 370 and the third stopper 380 will be omitted, as appropriate.

As illustrated in FIG. 4, a decoupling preventing large diameter portion 316 is provided in the vicinity of a proximal portion of the distal side coupling portion 310. When the distal side coupling portion 310 is moved toward the distal side, the decoupling preventing large diameter portion 316 is brought into contact with the support portion 362 of the first stopper 360, thereby preventing the distal side coupling portion 310 from moving toward the distal side with respect to the first stopper 360. The medical device 10 can prevent the distal side coupling portion 310 from being decoupled toward the distal side by the decoupling preventing large diameter portion 316.

As illustrated in FIG. 4, a first protruding portion 315 that can be fitted in a first recess portion 327 formed in an inner wall of the first coupling portion 320 is provided in the vicinity of the proximal portion of the distal side coupling portion 310.

In the medical device 10, movement of the distal side coupling portion 310 in the axial direction is restricted in a state in which the first protruding portion 315 is fitted in the first recess portion 327. For example, the distal side coupling portion 310 and the first coupling portion 320 can be held at respective predetermined initial positions by maintaining the first protruding portion 315 in a state of being fitted in the first recess portion 327 in a preparation stage or the like (a product shipping stage or a storing stage after manufacturing) before the medical device 10 is used in a medical setting. In the present embodiment, the first protruding portion 315 is provided on the distal side coupling portion 310 on the proximal side in the axial direction with respect to the large diameter portion 316, and the large diameter portion 316 may be provided on the distal side coupling portion 310 on the proximal side in the axial direction with respect to the first protruding portion 315.

The first recess portion 327 and the first protruding portion 315 are preferably configured to generate a fixing force that causes the first protruding portion 315 to be relatively easily decoupled from the first recess portion 327 when the operator applies a force that moves, relatively to the other of first protruding portion 315 and the first recess portion 327, one of the distal side coupling portion 310 and the first coupling portion 320 in the axial direction. The same applies to a second recess portion 337 and a second protruding portion 325 to be described below.

As illustrated in FIGS. 4 and 5, the first coupling portion 320 is configured of a tubular member including a lumen 323 allowing the puncture device 100 and the outer sheath 200 to penetrate (or extend through) the first coupling portion 320. A part of the distal side coupling portion 310 on the proximal side penetrates (or extends through) the lumen 323.

As illustrated in FIGS. 4 and 5, the second coupling portion 330 is configured of a tubular member including a lumen 333 allowing the inner sheath 140 and the outer sheath 200 to penetrate (or extend through) the second coupling portion 330. A part of the first coupling portion 320 on the proximal side penetrates (extends through) the lumen 333.

As illustrated in FIG. 5, a decoupling preventing large diameter portion 326 is provided at a proximal portion of the first coupling portion 320. When the first coupling portion 320 is moved toward the distal side, the decoupling preventing large diameter portion 326 is brought into contact with the support portion 362 of the second stopper 370, thereby preventing the first coupling portion 320 from moving toward the distal side with respect to the second stopper 370. The medical device 10 can help prevent the first coupling portion 320 from being decoupled toward the distal side by the decoupling preventing large diameter portion 326.

As illustrated in FIG. 5, the second protruding portion 325 that can be fitted in the second recess portion 337 formed in an inner wall of the second coupling portion 330 is provided in the vicinity of the proximal portion of the first coupling portion 320.

In the medical device 10, movement of the first coupling portion 320 in the axial direction is restricted in a state in which the second protruding portion 325 is fitted in the second recess portion 337. The first coupling portion 320 and the second coupling portion 330 can be held at respective predetermined initial positions by maintaining the second protruding portion 325 in a state of being fitted in the second recess portion 337 in a preparation stage or the like before the medical device 10 is used in a medical setting. In the present embodiment, the second protruding portion 325 is provided on the first coupling portion 320 on the proximal side in the axial direction with respect to the large diameter portion 326, and the large diameter portion 326 may be provided on the first coupling portion 320 on the proximal side in the axial direction with respect to the second protruding portion 325.

As illustrated in FIG. 5, the decoupling preventing large diameter portion 336 is provided at a proximal portion of the second coupling portion 330. When the third coupling portion 340 is moved toward the distal side, the decoupling preventing large diameter portion 336 is brought into contact with a distal portion 354 of the grip portion 350, thereby preventing the second coupling portion 330 from moving toward the distal side with respect to the distal portion 354 of the grip portion 350. The medical device 10 can prevent the second coupling portion 330 from being decoupled toward the distal side by the decoupling preventing large diameter portion 336.

As illustrated in FIGS. 4 and 5, the second stopper 370 is arranged on a distal side of the second coupling portion 330. The second stopper 370 has a configuration in which the second stopper is movable along an outer surface of the first coupling portion 320.

The second coupling portion 330 can be advanced and retracted in the axial direction along the outer surface of the first coupling portion 320. A maximum advance position (an advance limit position) of the second coupling portion 330 in the axial direction is set by a position at which the second stopper 370 is arranged. When the operator moves the second coupling portion 330 in the axial direction, the second coupling portion 330 can be advanced to a position at which the distal portion of the second coupling portion 330 is brought into contact with the second stopper 370. In addition, a projecting length of the inner sheath 140 coupled to the first coupling portion 320, the inner sheath protruding from the outer sheath 200, can be adjusted by the position of the second stopper 370.

As illustrated in FIG. 3, at least a part of the handheld handle portion 300 has the third coupling portion 340. The third coupling portion 340 is positioned on the proximal side with respect to the second coupling portion 330 and is coupled to the proximal portion 113 of the puncture needle 110. As illustrated in FIGS. 8 and 9, the third coupling portion 340 is configured to be advanced and retracted relatively to the second coupling portion 330, thereby causing the cutting edge portion 120 to protrude from the distal end of the inner sheath 140.

The handheld handle portion 300 has the grip portion 350 that can be gripped by an operator with fingers or the like. The third coupling portion 340 is arranged in the vicinity of a proximal portion of an internal space 353 of the grip portion 350. The proximal opening portion positioned at the proximal portion 113 of the puncture needle 110 coupled to the third coupling portion 340 is in fluid communication with the first port 351.

The third coupling portion 340 is coupled to the grip portion 350 together with the first port 351. In addition, the puncture needle 110 is coupled to the grip portion 350 via the third coupling portion 340.

The grip portion 350 can be advanced and retracted in the axial direction along an outer surface of the second coupling portion 330. When the grip portion 350 is moved in the axial direction along the outer surface of the second coupling portion 330, the third coupling portion 340 and the puncture needle 110 coupled to the grip portion 350 are moved in the axial direction in conjunction with the movement of the grip portion.

A protective member (a protective sheath) for covering and protecting a portion of the needle tube portion 130 of the puncture needle 110 exposed from the inner sheath 140 can be arranged, as appropriate, in the internal space 353 of the grip portion 350. In addition, a protective member (a protective sheath) for covering and protecting a portion of the inner sheath 140 exposed from the outer sheath 200 can be arranged, as appropriate, in the internal space of the lumen 333 which the inner sheath 140 and the outer sheath 200 have penetrated (extended through). The configuration of the protective member is not particularly limited as long as the puncture needle 110 can be prevented from being bent in the internal space 353 by manipulating the advancing and retreating of the handheld handle portion 300. For example, in the internal space 353 of the grip portion 350, the protective member may be a protective member that is configured of a tubular body having a small diameter and a tubular body having a large diameter which cover the external surface 130b of the needle tube portion 130, and enables the tubular body having a small diameter which has one end fixed to a wall of the internal space 353 of the grip portion 350 on the proximal side to penetrate (extend through) the tubular body having a large diameter which has one end fixed to a wall on the proximal side of the proximal portion 146 of the inner sheath 140 coupled to the second coupling portion 330. Alternatively, the protective member may be a protective member which is configured of two tubular bodies having the same diameter that cover the external surface 130b of the needle tube portion 130 and have engagement portions (for example, grooves, cuts) for engaging with each other and in which one end of each tubular body is fixed as described above and engagement is performed at the engagement portions. In the internal space of the lumen 333 which the inner sheath 140 and the outer sheath 200 have penetrated (or extended through), ends of two tubular bodies on one side are fixed to a wall on the proximal side of the proximal portion 206 of the outer sheath 200 and a wall on the distal side of the proximal portion 146 of the inner sheath 140 coupled to the second coupling portion 330, respectively. Here, the protective member may be fixed to penetrate (extend through), in the axial direction, the proximal portion 146 of the inner sheath 140 and the proximal portion 206 of the outer sheath 200 coupled to the second coupling portion 330.

As illustrated in FIGS. 3 and 5, the third stopper 380 is arranged on the distal side of the grip portion 350. The third stopper 380 has a configuration in which the third stopper is movable along an outer surface of the second coupling portion 330.

A maximum advance position (an advance limit position) of the grip portion 350 in the axial direction is set by a position at which the third stopper 380 is arranged. In other words, when the operator moves the grip portion 350 in the axial direction, the grip portion 350 can be advanced to a position at which the distal portion 354 of the grip portion 350 is brought into contact with the third stopper 380. In addition, a projection length of the puncture needle 110 that protrudes from the inner sheath 140 is adjusted by the position of the third stopper 380, the puncture needle 110 being coupled to the grip portion 350 via the third coupling portion 340.

The distal portion 354 of the grip portion 350 is preferably made of a resin material or a metal material having hardness to the extent that the grip portion 350 can be prevented from being damaged when the grip portion 350 is brought into contact with the third stopper 380.

As illustrated in FIG. 3, the medical device 10 has the first port 351 that is arranged to communicate with the needle tube lumen 135 via the third coupling portion 340 of the handheld handle portion 300 and enables a fluid to be supplied into the needle tube lumen 135.

The first port 351 can include, for example, a connector to which a liquid medicine supply instrument or an instrument capable of suctioning can be coupled.

### Example of Using Medical Device 10

Next, a method of administering the liquid medicine by using the medical device 10 will be described. The method of administering a liquid medicine to be described below is an example, and a procedure or the like of using the medical device 10 is not limited to the following details.

In the following description, a procedure for administering a liquid medicine to the target site E (for example, a region including biological tissue in which cancer cells are present) of a pancreas M by using the medical device 10 will be described. Here, as illustrated in FIG. 13, a procedure of using the endoscopic device 500 to deliver the medical device 10 through a stomach and deliver the puncture needle 110 to the pancreas M through a stomach wall W will be described.

When the operator starts liquid medicine administration using the medical device 10, the operator inserts the endoscopic device 500 into the living body of the patient. The operator delivers the insertion portion 510 of the endoscopic device 500 to a predetermined position in the stomach, and then the operator checks a state of the target site E by using the ultrasound image pickup unit 520.

The operator couples a liquid medicine supply instrument to the first port 351 (see FIG. 3) and feeds the liquid medicine to the handheld handle portion 300, thereby performing priming of the medical device 10.

The operator manipulates the first stopper 360 and the distal side coupling portion 310 to adjust an effective length of the medical device 10 to correspond to an effective length of the insertion portion 510 of the endoscopic device 500.

The operator inserts the medical device 10 into the channel 511 of the insertion portion 510 of the endoscopic device 500 and delivers the medical device 10 to the stomach via the insertion portion 510. While the operator moves the medical device 10 in the channel 511, the operator maintains a state in which the one or more hole portions 136 are covered with the inner sheath 140 (the state illustrated in FIG. 8), with the cutting edge portion 120 of the puncture needle 110 projecting from the inner sheath 140. In addition, while the medical device 10 is delivered to the stomach, the operator maintains a state in which the cutting edge portion 120 of the puncture needle 110 and the distal portion of the inner sheath 140 are accommodated in the first lumen 203 of the outer sheath 200 (a state in which the cutting edge portion 120 of the puncture needle 110 and the distal portion of the inner sheath 140 do not project from the outer sheath 200).

In the state in which the puncture device 100 is covered with the outer sheath 200 as described above, the operator can deliver the medical device 10 to a desired position in the living body via the channel 511 of the endoscopic device 500. Regarding the medical device 10, an outer diameter of a portion of the medical device 10 inserted into the channel 511 is increased by an amount corresponding to an outer diameter of the outer sheath 200 as compared with a case where the outer sheath 200 is not provided. Therefore, as illustrated in FIG. 6, the operator can narrow a clearance formed between the medical device 10 and an inner wall of the insertion portion 510 while moving the medical device 10 in the channel 511. In FIG. 6, the insertion portion 510 of the endoscopic device 500 is illustrated by a pair of dashed lines, with the insertion portion 510 shown as the outer dashed line and the channel 511 shown as the inner dashed line.

If an excessive clearance is formed between the medical device 10 and the inner wall of the insertion portion 510 while the medical device 10 is moved in the channel 511, the medical device 10 is likely to be distorted or bent. When the medical device 10 is distorted or bent, the distal portion of the medical device 10 protruding from the insertion portion 510 of the endoscopic device 500 results in entering the state of protruding from the distal end of the insertion portion 510 to be largely separated from a central axis of the channel 511. This results in a positional displacement in a radial direction around a central axis of the insertion portion 510, between the proximal portion of the puncture device 100 positioned in the vicinity of a hand side of the endoscopic device 500 (in the vicinity of an insertion port of the endoscopic device 500) and the vicinity of the distal portion of the medical device 10 protruding from the insertion portion 510 of the endoscopic device 500. When such positional displacement occurs, the manipulability of the medical device 10 deteriorates (torque transmission performance is not sufficient), it becomes difficult to locate, by the ultrasound image pickup unit 520, the position of the cutting edge portion 120 positioned at the distal portion of the puncture device 100, or the pushability of the puncture device 100 deteriorates, and the pushing force applied on the proximal portion side of the medical device 10 cannot be sufficiently transmitted to the cutting edge portion 120. As described above, the medical device 10 according to the present embodiment can prevent an excessive clearance from being formed between the medical device 10 and the inner wall of the insertion portion 510, and thus, it is possible to favorably prevent the above-described problem from occurring.

In addition, in the medical device 10, the second coupling portion 330 of the handheld handle portion 300 is advanced and retracted relatively with respect to the first coupling portion 320 of the handheld handle portion 300 coupled to the outer sheath 200, thereby enabling the projection of the puncture device 100 from the first lumen 203 (see FIG. 6) of the outer sheath 200 and the accommodation of the puncture device 100 in the first lumen 203 of the outer sheath 200 to be manipulated. Therefore, the operator can smoothly perform a predetermined operation using the medical device 10 (the delivery of the medical device 10 to the target site E or local administration of the liquid medicine to the target site E).

In addition, the outer sheath 200 includes two lumens of the first lumen 203 enabling the puncture device 100 to penetrate (extend through) and the second lumen 204 positioned outward in a radial direction of the first lumen 203. The outer sheath 200 is configured to have moderate flexibility by including the two lumens 203 and 204. Therefore, the operator can relatively smoothly deliver the medical device 10 to the desired position in the living body via the channel 511 of the insertion portion 510.

As illustrated in FIG. 14, the operator can cause the puncture needle 110 and the inner sheath 140 to protrude from the insertion portion 510 via a distal opening portion of the insertion portion 510 by moving the medical device 10 to the distal portion of the endoscopic device 500. As illustrated in FIG. 16, the operator moves the puncture device 100 toward the target site E of the pancreas M in a state in which the cutting edge portion 120 of the puncture needle 110 protruding from the inner sheath 140. At this time, the operator causes the puncture needle 110 to puncture the stomach wall W and further penetrate the stomach wall W in a state in which the one or more hole portions 136 are covered with the inner sheath 140 (the state illustrated in FIG. 8) while the cutting edge portion 120 of the puncture needle 110 projects from the inner sheath 140. The operator covers the one or more hole portions 136 with the inner sheath 140 as described above, thereby enabling the biological tissue to be prevented from entering the hole portion 136 and enabling the one or more hole portions 136 to be prevented from being blocked (clogged) with the biological tissue.

The cutting edge portion 120 of the puncture needle 110 has the plurality of cutting surfaces 121 and the plurality of ridge lines 123 positioned at boundaries between the plurality of cutting surfaces 121. Therefore, the operator manipulates the cutting edge portion 120 to be pushed into the biological tissue, thereby enabling the cutting edge portion 120 to be smoothly inserted while cutting and opening the biological tissue by the plurality of cutting surfaces 121 and the plurality of ridge lines 123.

The one or more hole portions 136 of the puncture needle 110 are formed in the side surface of the needle tube portion 130. Therefore, when the cutting edge portion 120 is inserted into the biological tissue, the biological tissue can be prevented from entering the hole portion 136 and clogging the hole portion 136. Hence, the puncture needle 110 can help prevent the injection resistance of the liquid medicine from increasing as the hole portion 136 is clogged.

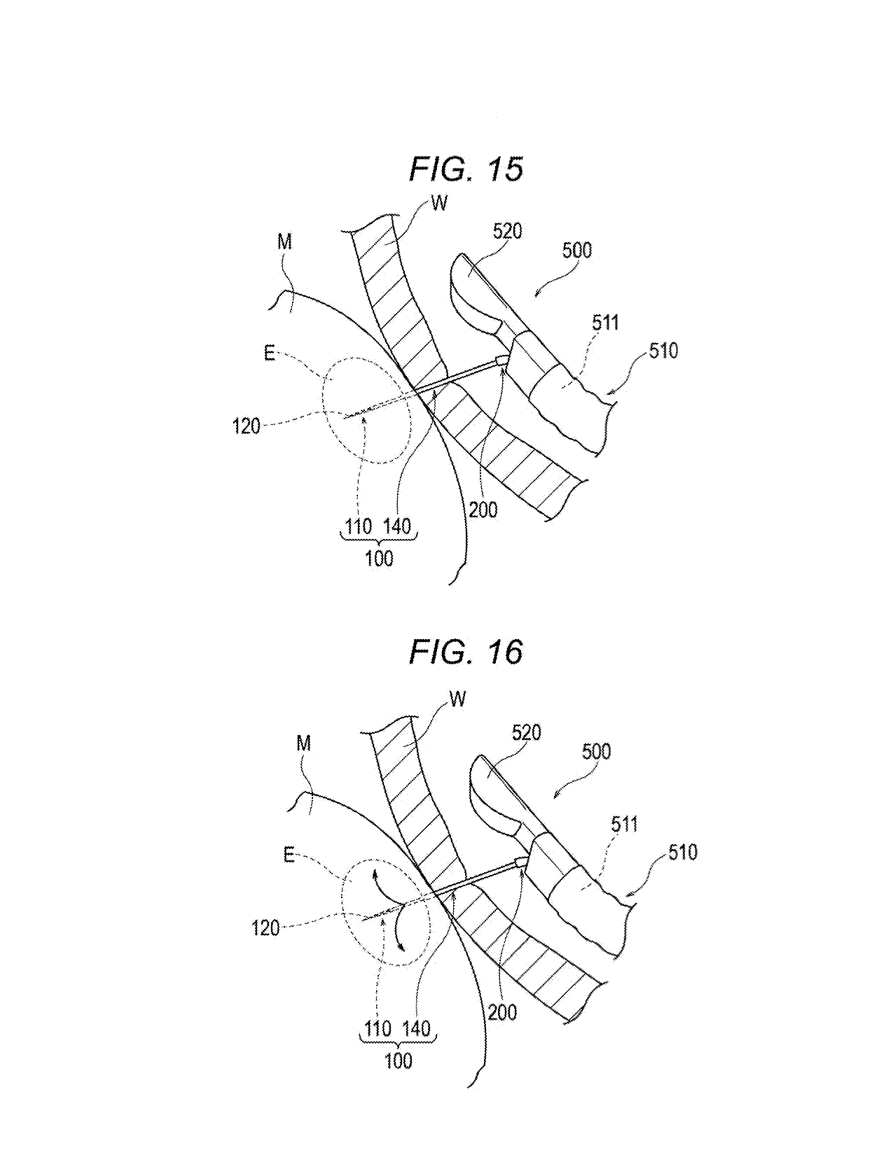

As illustrated in FIG. 15, the operator makes the cutting edge portion 120 and the needle tube portion 130 penetrate the stomach wall W and then advances the cutting edge portion 120 and the needle tube portion 130 with respect to the inner sheath 140, thereby causing the portion where the cutting edge portion 120 and the one or more hole portions 136 of the needle tube portion 130 are formed to enter the state of protruding from the inner sheath 140 (the state illustrated in FIG. 9). The operator makes the cutting edge portion 120 and the needle tube portion 130 protrude from the inner sheath 140 as described above, thereby inserting a certain range on the distal side of the puncture needle 110 (a certain range including at least a portion on the proximal side with respect to the position where the hole portion 136 of the needle tube portion 130 is formed) to the target site E of the pancreas M.

As illustrated in FIG. 16, the operator supplies the liquid medicine to the needle tube lumen 135 via the first port 351 in a state in which the one or more hole portions 136 of the needle tube portion 130 are arranged at the target site E. The operator can locally administer the liquid medicine to the target site E by discharging the liquid medicine supplied to the needle tube lumen 135 to the outside of the needle tube portion 130 via the one or more hole portions 136. Examples of the liquid medicine used in the procedure described here can include a CAR-T cell, an mRNA drug, an anticancer drug, an immune checkpoint inhibitor, or the like. The liquid medicine that becomes an administration target can be selected arbitrarily depending on the details of a procedure using the medical device 10, a biological organ that becomes a medicine using target, a disorder that becomes a medical treatment target, and the like.

The puncture needle 110 includes two or more hole portions 136 formed in the side surface of the needle tube portion 130 (see FIG. 12). Therefore, the operator can efficiently administer the liquid medicine to the target site E via the needle tube lumen 135 and the two or more hole portions 136.

In addition, in the puncture needle 110, the one or more hole portions 136 are arranged on extension of the ridge lines 123 in the axial direction (see FIG. 9). The liquid medicine discharged from the one or more hole portions 136 moves in the axial direction along the needle tube portion 130 and the external surface of the cutting edge portion 120 and reaches the ridge lines 123. The ridge lines 123 of the cutting edge portion 120 exhibit a function of cutting and opening the biological tissue when the cutting edge portion 120 is inserted into the biological tissue. Therefore, the liquid medicine moved from the hole portion 136 to the ridge line 123 efficiently diffuses along an incision surface formed by the ridge line 123. Therefore, the operator can more efficiently administer the liquid medicine to the target site E.

After the administration of the liquid medicine to the target site E is completed, the operator removes the puncture needle 110 from the pancreas M.

The operator accommodates the puncture needle 110 removed from the pancreas M in the inner sheath 140, and the state illustrated in FIG. 7 is obtained. The operator accommodates the puncture needle 110 and the inner sheath 140 in the outer sheath 200 in a state in which the puncture needle 110 is accommodated in the inner sheath 140.

In a state in which the puncture needle 110 and the inner sheath 140 are accommodated in the outer sheath 200, the operator accommodates the medical device 10 in the insertion portion 510 of the endoscopic device 500 and removes the medical device 10 from the living body via the channel 511 of the insertion portion 510. After the operator punctures the target site E with the puncture needle 110, the operator performs work of accommodating the puncture needle 110 in the inner sheath 140 and further removing the medical device 10 out of the living body in a state in which the inner sheath 140 is accommodated in the outer sheath 200, thereby enabling the biological tissue (for example, the biological tissue of a lesion area) attached to the puncture needle 110 that has punctured the target site E to be prevented from being disseminated in the living body.

The operator can locally administer the liquid medicine to the target site E by using the medical device 10 according to the procedure described above.

Hereinafter, operations and effects of the puncture needle 110 according to the present embodiment will be described.

The puncture needle 110 includes the needle tube portion 130 having the needle tube lumen 135 extending in the axial direction, the cutting edge portion 120 having the plurality of cutting surfaces 121 formed at different positions around an axis and the plurality of ridge lines 123 positioned at respective boundaries of the plurality of cutting surfaces 121, the cutting edge portion being arranged on the distal side of the needle tube portion 130, and the one or more hole portions 136 that are formed in the side surface of the needle tube portion 130 and allow the needle tube lumen 135 of the needle tube portion 130 to communicate with the outside. The one or more hole portions 136 are arranged on extension of the ridge lines 123 in the axial direction, and two or more of the hole portions 136 are provided.

Since the puncture needle 110 includes the two or more hole portions 136 formed in the side surface of the needle tube portion 130, the liquid medicine can be efficiently administered to the target site E via the two or more hole portions 136. In addition, in the puncture needle 110, since the one or more hole portions 136 are arranged on the extension of the ridge lines 123 in the axial direction, the liquid medicine moved from the one or more hole portions 136 to the ridge lines 123 can efficiently diffuse along the incision surface formed by the ridge lines 123. Therefore, even in a case where the puncture needle 110 has a relatively small diameter, the liquid medicine can be efficiently injected into the target site E, and the puncture needle 110 can have high insertability into the biological tissue.

In addition, in the puncture needle 110, at least on the proximal side of the hole portion 136, the inner wall of the needle tube portion 130 having the one or more hole portions 136 formed on the inner side thereof intersects the external surface 130b of the needle tube portion 130 at an obtuse angle.

As described above, since at least on the proximal side of the one or more hole portions 136, the inner wall of the needle tube portion 130 having the one or more hole portions 136 formed on the inner side of the inner wall of the needle tube portion 130 intersects the external surface 130b of the needle tube portion 130 at an obtuse angle, the puncture needle 110 can prevent the biological tissue from being caught on the inner wall around the one or more hole portions 136 of the needle tube portion 130 when the needle tube portion 130 is inserted into biological tissue. Therefore, it is possible to effectively help prevent the one or more hole portions 136 from being clogged with the biological tissue. In addition, since the liquid medicine flowing into the needle tube lumen 135 can flow along the inner wall of the needle tube portion 130 at least on the proximal side of the one or more hole portions 136, the liquid medicine can be efficiently discharged to the outside of the needle tube portion 130 (the outside of the puncture needle 110) via the one or more hole portions 136 while reducing the injection resistance at the time of liquid medicine administration.

In addition, the hole portion 136 has a substantially rectangular shape having a long side extending along the axial direction in plan view.

Since the puncture needle 110 has a configuration in which the hole portion 136 is formed in the substantially rectangular shape having a long side in the axial direction, the liquid medicine can be discharged over a relatively wide range in the axial direction when the liquid medicine is administered to the target site E. This enables the liquid medicine to be efficiently discharged while reducing the injection resistance at the time of liquid medicine administration.

In addition, the corner portions 136a of hole portion 136 are chamfered.

In the puncture needle 110, since the corner portions 136a of the hole portion 136 are chamfered, the liquid medicine diffuses to spread around via the corner portions 136a when the liquid medicine is discharged from the hole portion 136. Therefore, the amount of administration of the liquid medicine can be increased while reducing the injection resistance. In addition, when the needle tube portion 130 is inserted into the biological tissue, the biological tissue is unlikely to be caught by the chamfered corner portions 136a of the hole portion 136, and the hole portion 136 can be more effectively prevented from being clogged with the biological tissue.

In addition, the groove portion 138 extending from the hole portion 136 toward the ridge line 123 is formed in the external surface 130b of the needle tube portion 130.

In the puncture needle 110, the liquid medicine discharged from the hole portion 136 is guided toward the ridge line 123 via the groove portion 138. Therefore, in the puncture needle 110, the liquid medicine can diffuse from the periphery of the hole portion 136 over a wide range on the distal side where the ridge line 123 is positioned. Therefore, the puncture needle 110 can more efficiently administer the liquid medicine to the target site E.

In addition, the distal side end portion of the groove portion 138 extends to at least the boundary position 105 between the cutting edge portion 120 and the needle tube portion 130.

In the puncture needle 110, since the groove portion 138 extends to the boundary position 105 between the cutting edge portion 120 and the needle tube portion 130, the liquid medicine discharged from the hole portion 136 can be more efficiently guided toward the ridge line 123.

The cutting edge portion 120 has the solid structure and is fixed to the needle tube portion 130 on the distal side of the needle tube portion 130.

Since the cutting edge portion 120 has a central-axis structure, the insertability of the cutting edge portion 120 into the biological tissue can be enhanced. In addition, since the puncture needle 110 has a configuration in which two members of the needle tube portion 130 in which the needle tube lumen 135 is formed and the cutting edge portion 120 in which the needle tube lumen 135 is not formed are fixed, it is possible to impart different functions (a function of improving the insertability to the cutting edge portion 120, and a function of delivering the liquid medicine to the needle tube portion 130) to the cutting edge portion 120 and the needle tube portion 130, respectively.

In addition, the cutting edge portion 120 has ridge lines 123 as many as the number of the one or more hole portions 136.

The puncture needle 110 can guide the liquid medicine from the individual hole portions 136 toward the corresponding number of ridge lines 123. Therefore, the puncture needle 110 can more efficiently administer the liquid medicine to the target site E.

Next, a modification example of the above-described embodiment will be described. In the description of the modification example, detailed descriptions of the members, the use procedure of the puncture needle, and the like already described will be omitted, as appropriate. In addition, details that are not particularly described in the modification example can be the same as those in the above-described embodiment.

### Modification Example

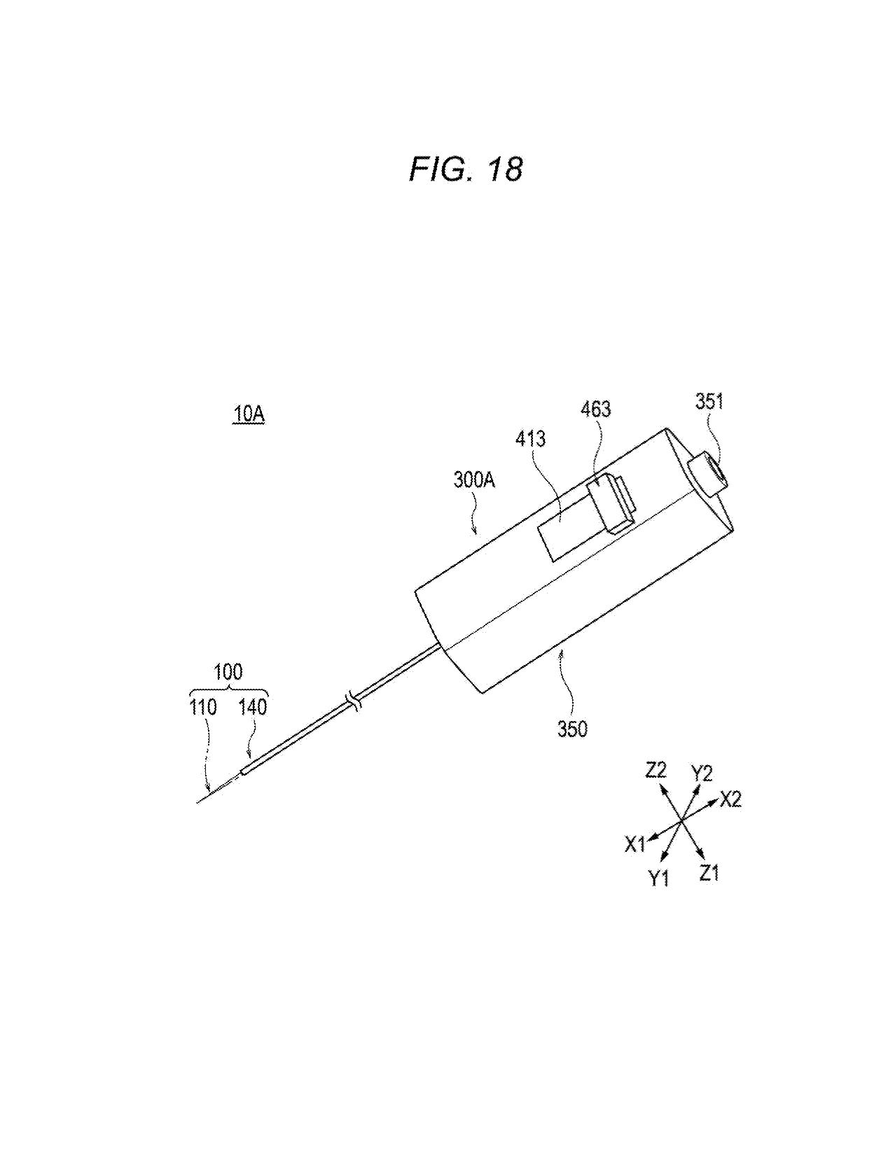

FIG. 18 illustrates a medical device 10A according to a modification example.

A specific usage state or the like of the puncture needle 110 according to the present embodiment is not particularly limited as long as the puncture needle is used for the purpose of administering a liquid medicine to a predetermined target site E. For example, in the above-described embodiment, an example of a device configuration in which the puncture device 100 including the puncture needle 110 can be delivered to a desired position in the living body by using the delivery device (the endoscopic device 500) has been described. However, the puncture device 100 is not limited to the device that is assumed to be used in a procedure using the delivery device in combination.

The medical device 10A according to the modification example illustrated in FIG. 18 is configured as a device for percutaneous puncture administration for realizing local liquid medicine administration to the predetermined target site E in the living body.

In a procedure using the medical device 10A according to the present modification example, the operator can use a device for acquiring a diagnostic image such as a CT device instead of an image pickup unit of the delivery device, for example, when checking a lesion area or a puncture route.

Since the medical device 10A is based on percutaneous puncture, the medical device 10A does not include the outer sheath 200 (see FIG. 1) that can be inserted into the insertion portion 510 of the delivery device such as the endoscopic device 500.

The medical device 10A includes a handheld handle portion 300A for enabling the puncture needle 110 to protrude from the inner sheath 140 and the puncture needle 110 to be accommodated in the inner sheath 140.

The handheld handle portion 300A includes a grip portion 350 having a rectangular parallelepiped shape. The grip portion 350 has a knob portion 463 for manipulating the advancing and retracting of the puncture needle 110 in the axial direction. The knob portion 463 is coupled to the puncture needle 110 and is structured to be slidable along a slide hole 413 provided in the grip portion 350. When the puncture needle 110 is advanced and retracted, the operator can manipulate the movement of the knob portion 463 by gripping the knob portion 463 with fingers or the like. The grip portion 350 of the present modification example has a rectangular parallelepiped shape, but the shape of the grip portion 350 is not particularly limited as long as the grip portion 350 has a configuration in which the puncture needle 110 can protrude from the inner sheath 140 and the puncture needle 110 can be accommodated in the inner sheath 140. For example, the grip portion 350 may have a cylindrical shape or a tapered cylindrical shape.

The first port 351 configured to be in fluid communication with the needle tube lumen 135 of the puncture needle 110 is provided at a proximal portion of the grip portion 350. The first port 351 of the present modification example is provided at a proximal end of the grip portion 350 and may be provided on a side surface of the grip portion 350 on the distal side with respect to the proximal end as long as the first port 351 is configured to be in fluid communication with the needle tube lumen 135 of the puncture needle 110.

The operator can control the movement of the puncture needle 110 in the axial direction by gripping the knob portion 463 with fingers or the like and advancing and retracting the knob portion 463 in the axial direction. In a procedure using the puncture needle 110, the operator performs manipulation of appropriately switching between the projection of the puncture needle 110 from the inner sheath 140 and the accommodation of the puncture needle 110 in the inner sheath 140, thereby enabling the biological tissue of the target site E attached to the puncture needle 110 to be favorably prevented from being disseminated in the living body, similarly to the above-described embodiment.

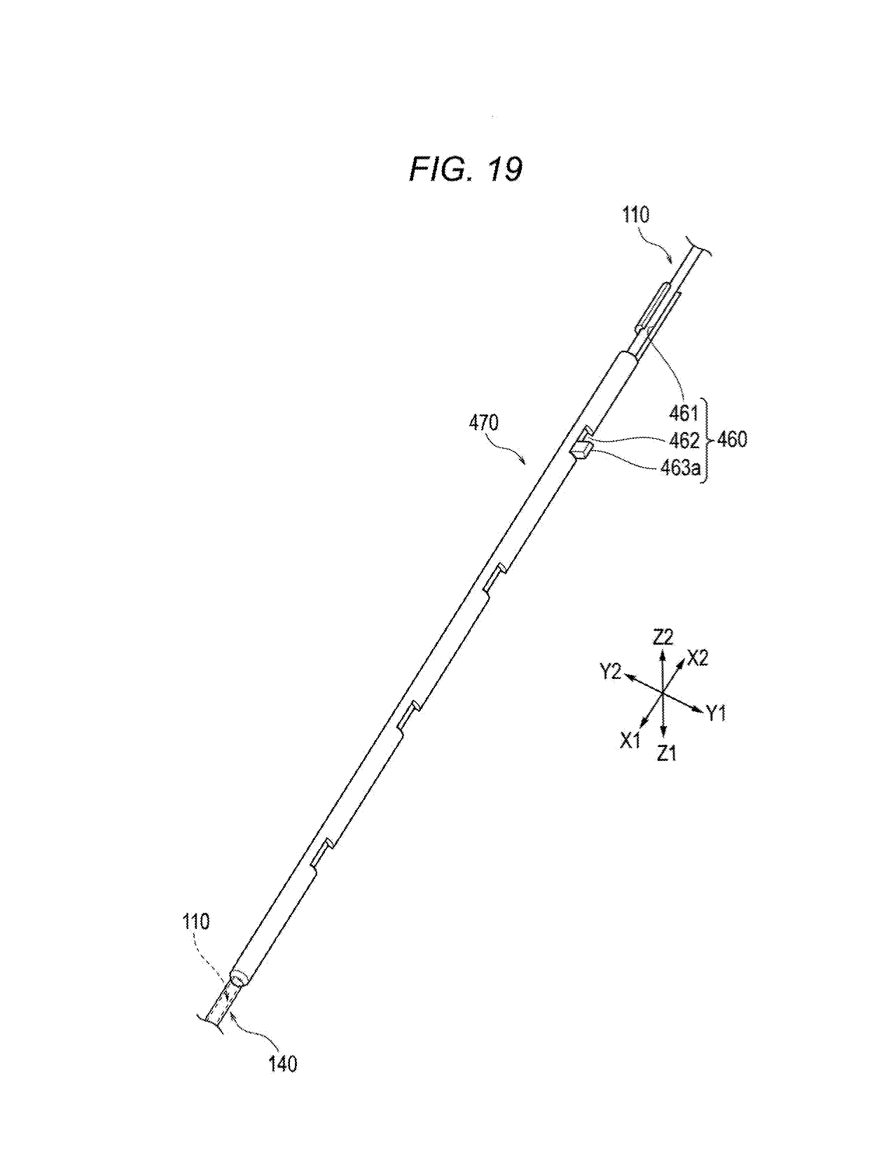

A lock mechanism 460 illustrated in FIG. 19 can be added to the handheld handle portion 300A according to the modification example, as appropriate.

The lock mechanism 460 can be provided in a predetermined protective sheath 470. The inner sheath 140 can penetrate (or extend through) the protective sheath 470. The protective sheath 470 has a first groove portion 461 and a second groove portion 462. In the vicinity of the proximal portion 113 of the puncture needle 110 accommodated in the inner sheath 140, a knob portion 463a that is movable along each of the groove portions 461 and 462 and is coupled to the puncture needle 110 is installed. The lock mechanism 460 includes the first groove portion 461, the second groove portion 462, and the knob portion 463a. The protective sheath 470 can be arranged in the vicinity of the proximal portion of the puncture needle 110 arranged in the grip portion 350.

The first groove portion 461 extends in a straight line shape in an axial direction of the protective sheath 470, and the second groove portion 462 extends in a direction intersecting the first groove portion 461 (in a circumferential direction of the inner sheath 140). A plurality of the second groove portions 462 are arranged at intervals in the axial direction of the protective sheath 470.

The cutting edge portion 120 is arranged at a position where the cutting edge portion 120 is accommodated in the inner sheath 140 in a state in which the knob portion 463a is arranged at a proximal portion of the protective sheath 470. In the case of maintaining a state in which the cutting edge portion 120 does not project or protrude from the inner sheath 140, the operator grips the knob portion 463 with fingers or the like and moves the knob portion 463a into the second groove portion 462 so that the knob portion enters a lock state. In the lock state, the knob portion 463a can be restricted from moving in the axial direction along the first groove portion 461. Therefore, the operator can help prevent the cutting edge portion 120 from inadvertently projecting or protruding from the inner sheath 140.

When the operator causes the cutting edge portion 120 to protrude from the inner sheath 140, the operator grips the knob portion 463a with fingers or the like and moves the knob portion 463a in the lock state from the second groove portion 462 to the first groove portion 461. The operator can further move the knob portion 463a along the first groove portion 461, thereby moving the puncture needle 110 in the axial direction in conjunction with the knob portion 463a.

In the protective sheath 470, a plurality of second groove portions 462 are arranged at intervals in the axial direction. Therefore, the operator can switch to the lock state by using an arbitrary second groove portion 462, thereby adjusting, in multiple stages, the projection length of the cutting edge portion 120 protruding from the inner sheath 140.

In the above-described example, the configuration in which the movement of the puncture needle 110 in the axial direction is controlled by the lock mechanism 460 has been exemplified. However, for example, a mechanism for controlling the movement of the puncture needle 110 and/or the inner sheath 140 in the axial direction can be additionally provided to the medical device 10 by being installed in a similar configuration to the medical device 10 of the above-described embodiment (see FIG. 1).

The detailed description above describes embodiments of a puncture needle. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

### Reference Signs List

10 medical device

10A medical device

100 puncture device

105 boundary position

110 puncture needle

113 proximal portion of puncture needle

120 cutting edge portion

121 cutting surface

123 ridge line

125 cylindrical portion

130 needle tube portion

130a inner wall of needle tube portion

130b external surface of needle tube portion

135 needle tube lumen

136 hole portion

136a corner portion of hole portion

138 groove portion

140 inner sheath

200 outer sheath

210 inner tube

220 outer tube

250 fixing member

300 handheld handle portion

460 lock mechanism

500 endoscopic device (delivery device)

510 insertion portion

511 channel

520 ultrasound image pickup unit

C1 central axis of inner sheath

C2 central axis of outer sheath

C3 central axis of puncture needle

g clearance

E target site

M pancreas

W stomach wall

What is claimed is:

1. A puncture needle comprising:
a needle tube portion having a needle tube lumen extending in an axial direction;
a cutting edge portion having a plurality of cutting surfaces formed at different positions around an axis and a plurality of ridge lines positioned at respective boundaries of the plurality of cutting surfaces, the cutting edge portion being arranged on a distal side of the needle tube portion; and
one or more hole portions that are formed on a side surface of the needle tube portion and allow the needle tube lumen of the needle tube portion to communicate with an outside of the needle tube portion, wherein the one or more hole portions are arranged on an extension of each of the plurality of ridge lines in the axial direction.

2. The puncture needle according to claim 1, wherein on at least a proximal side of the one or more hole portions, an inner wall of the needle tube portion in which the hole portions are formed intersects an external surface of the needle tube portion at an obtuse angle.

3. The puncture needle according to claim 1, wherein each of the one or more hole portions has, in plan view, a substantially rectangular shape having a long side extending along the axial direction.

4. The puncture needle according to claim 3, wherein a corner portion of each of the one or more hole portions is chamfered.

5. The puncture needle according to claim 1, wherein a groove portion extending from each of the one or more hole portions toward each of the ridge lines is formed in an external surface of the needle tube portion.

6. The puncture needle according to claim 5, wherein a distal side end portion of the groove portion extends to at least a boundary position between the cutting edge portion and the needle tube portion.

7. The puncture needle according to claim 1, wherein the cutting edge portion has a solid structure and is fixed to the needle tube portion on the distal side of the needle tube portion.

8. The puncture needle according to claim 1, wherein the cutting edge portion has a number of the plurality of ridge lines, which is equal to a number of the one or more hole portions.

9. The puncture needle according to claim 8, wherein the number of the plurality of ridge lines and the one or more hole portions is four.

10. A puncture needle comprising:
a needle tube portion having a needle tube lumen extending in an axial direction;
a cutting edge portion having a plurality of cutting surfaces formed at different positions around an axis, the cutting edge portion being arranged on a distal side of the needle tube portion; and
one or more hole portions that are formed on a side surface of the needle tube portion and allow the needle tube lumen of the needle tube portion to communicate with an outside of the needle tube portion, the one or more hole portions being arranged on an extension of each of the plurality of cutting surfaces in the axial direction.

11. The puncture needle according to claim 10, wherein on at least a proximal side of the one or more hole portions, an inner wall of the needle tube portion in which the hole portions are formed intersects an external surface of the needle tube portion at an obtuse angle.

12. The puncture needle according to claim 10, wherein each of the one or more hole portions has, in plan view, a substantially rectangular shape having a long side extending along the axial direction.

13. The puncture needle according to claim 12, wherein a corner portion of each of the one or more hole portions is chamfered.

14. The puncture needle according to claim 10, further comprising:
a groove portion extending from each of the one or more hole portions toward each of the plurality of cutting surfaces formed in an external surface of the needle tube portion; and
a distal side end portion of the groove portion extends to at least a boundary position between the cutting edge portion and the needle tube portion.

15. The puncture needle according to claim 10, wherein the cutting edge portion has a solid structure and is fixed to the needle tube portion on the distal side of the needle tube portion.

16. A medical device that is retractably inserted into a delivery device, the medical device comprising:
an outer sheath; and
a puncture needle that is configured to be inserted into the outer sheath, the puncture needle including a needle tube portion having a needle tube lumen extending in an axial direction, a cutting edge portion, and a one or more hole portions that are formed on a side surface of the needle tube portion and allow the needle tube lumen of the needle tube portion to communicate with an outside of the needle tube portion.

17. The medical device according to claim 16, wherein the cutting edge portion includes a plurality of cutting surfaces formed at different positions around an axis and a plurality of ridge lines positioned at respective boundaries of the plurality of cutting surfaces, the cutting edge portion being arranged on a distal side of the needle tube portion; and
wherein the one or more hole portions are arranged on an extension of each of the plurality of ridge lines in the axial direction.

18. The medical device according to claim 17, further comprising:
a groove portion extending from each of the one or more hole portions toward each of the ridge lines is formed in an external surface of the needle tube portion, and wherein a distal side end portion of the groove portion extends to at least a boundary position between the cutting edge portion and the needle tube portion.

19. The medical device according to claim 16, wherein on at least a proximal side of the one or more hole portions, an inner wall of the needle tube portion in which the hole portions are formed intersects an external surface of the needle tube portion at an obtuse angle.

20. The medical device according to claim 16, wherein each of the one or more hole portions has, in plan view, a substantially rectangular shape having a long side extending along the axial direction, and a corner portion of each of the one or more hole portions is chamfered.
